SEQUENCE LISTING (26 sequences)

SEQ ID NO: 1 - DNA, 809 bp, unidentified
  source 1..809: mol_type = other DNA; note = Description of Unknown: Reoviridae family sequence
gcataaaatggcgttaacgctgcttctcgtccttgctgcttgcgctaatgcacaattgaatgttattccagcaactgatcctgaaatatgcatactatatgctgatgatatgaatgacgcgaaacaatattttgggaattttacagagatttttgaaagttataatcacgtaactataagctttacgaactattcatccgataactatgacgttattgaaatcttatccaaatataactatgatgcctgcgatatactagccatttatgtaaagtatgagtatatggattttgcaacttttctgcaatctgaaaacaactgttccaaatttgcaggtgggaaaattcactatgtgcaattaccaagagaccaagaatggttcgtgtattcaaaagatctcaagttttgtccattgtcagacgatctgatagggatgtattgtgatacacaattatctgggacctattttgaggtagctccaaacagaagatactacgtcacggacatcccagaattcactagtaaaggttacaccttatattctaacaatccattttacgtatgtcaaagaatcacagaaaacccatggattaacgtacattatttttatgctggcaatgaaccatcagggacaatttctaaacgaataagttggggtaatgtctggacaaatgtcacaacattcgcgcaaatgttatataaaatattagatatattcttcaatagtagtagaagtgctcaaccgcgagcttaaagaggactaggcgaaagggaggaaaccaaacgagtagctgaagcaaataaaaacc

SEQ ID NO: 2 - protein, 248 aa, unidentified
  source 1..248: note = Description of Unknown: Reoviridae family sequence; note = VP7
MALTLLLVLAACANAQLNVIPATDPEICILYADDMNDAKQYFGNFTEIFESYNHVTISFTNYSSDNYDVIEILSKYNYDACDILAIYVKYEYMDFATFLQSENNCSKFAGGKIHYVQLPRDQEWFVYSKDLKFCPLSDDLIGMYCDTQLSGTYFEVAPNRRYYVTDIPEFTSKGYTLYSNNPFYVCQRITENPWINVHYFYAGNEPSGTISKRISWGNVWTNVTTFAQMLYKILDIFFNSSRSAQPRA

SEQ ID NO: 3 - DNA, 3442 bp, unidentified
  source 1..3442: mol_type = other DNA; note = Description of Unknown: Reoviridae family sequence
ccagcttagtgtatacaaatccaaaggtagcaatagttcaattcaccagaactgataacgaaaaattatggcagtcaaaagaattgaacgtgttatyaccagtagaattactaacgcaactaaagaatgaactagatagagcaaaaacgctagatgaaaaaattgaaatacttttaagattaagatacttcactgtatatgtcgaagacaaatctgataaaagatcaattatatcaagctggctaagaaaaactataacagaattgggtgatgaacccgagtttgagtcaatacaactcattgaacatcaagctcgacaatggaaaatcgacaacgcaggctctctaagagcatatcatcacaacatccctatcaatgaattcattcgtgataatgaaattgaaataattgacacaggagattataaatggaagtcggacacactggcaggattaaacccaaactattatcacagaacgcacacgttaattgggtcagttcttttcgcgattcactcaagacttaaattctatactggagacaaaaaaagagcacttgcatatttgttaaaggtaatagaacaatgttattcccagggctatcttgaattatcaagaaatagaaaatggtctcacactatcaatgaattgcgcaagtctaattttagattatataatgcaaaagtgatacatgcggcatgcgctatgatatccctactacatgcggatccgatcaacgcagaattcttatgtcaaataattgcagtgtaccagatcatgccttcacatgcagcaaaagtattgtcatctccgatgacgttatatgtaggtatcgcaaccttcccatccagacaagtaatatcaactggtaatgcttcagagtgtgccccgacatcgaatccaaataacacttttgttgctgaatcacaaaagaatgtatggcgtgaagcttataagaatgatcctctgaatcactccaaaatgttagagattatgaatctgaatctaacaacaaacgttggaactttctcactgatatttaattgtttttcagcaacgtttcatgttggccatagagtagacaatgcccaagatgctataacagaacaagtttcagttaaatacacctcagatgttgacaaagaaatgtatgacacatactactataaattgaaacaaatgctgaaagacgaaattattcagtatgtggaagaacacatagcaaagaactatagggacgtaactgcagaatctttgtcagctctagccaattcatcaaacggattcattaaagaagttgagtttgtggatagaaaaataaaaacgacgaagaagattttacatctcgattctgacttgatttcaaatacatacagtgaccttactaaatcactatcacatgggataccaatgggtaccagaaacgttccagctagacaaacgagaggtattttcatattaccttggcaagtggcggccattcaacatacaattgcagagtcattatacaaaagagcaaaaaaaggagcatatggaggatcgtttgctgaagcgtatacagcaaaaaccgcttctttaacttatggtgtgttagctgaagatacgtcaaaagcaatgaggatcatactttatactgatgtgtctcaatgggatgctagtcaacataacactgtgccataccgttcggcatggataaatgctatacgtgaagcgagatcggaattacgctggaactattcagatgaaccatacgttttaggtatgaatgttttagacaatatggttaagatacaagaatatctactaaactctaacctggttgtatcatctccaggatcattgagaccaacaaaaatcattcgataccatggtgtagcctctggtgagaaaactacaaaaattggaaattcatttgcaaacgttgcattgatcgaaacagtcctagactatacaaaacaacaaataccagatcttgaaatttcacatctaagagttgacggtgacgacaatgttgtaactattaatacatcatgtccaattgagagattacaagccataattaaaaacaactactcgaagttaaacgctagagtgaaagcactagcatcatatacaggactcgaaatggcaaagagatttgttgtatgcggcaaaatatttgagagaggtgcgattccaatatttactgctgaaaggccctatggtacagatctatcaacacagtctatgtgtggatcttcaatctattcaacggccgtaaacgcatatcgagggtttggcgatgaatatttttcatttatgctagatgtactggttccaccttcatcatcagttagagtcacagggagattacgagtgttgttatcaccagttacattatatgcgactggtcctttaagctttgaagttacgccgcaaggtttaggaggtagaggaagaatgttcacagaatctaaaaaattgttcacactatttaaattacttacacaaacagtatcagtgtcggtcacgccagaggatatcaaaaaatattcagctactaaacagtttaatgcgcgaactgacgtcatgattaaaagcatgcgtgagagagtaggtggggatgcaaaggcattaaacagaataatgattgacaaggaagaacagaagactctaggtgtaccgaacgttttaagtcaaaaaaatagagatcaagtatcacaagctatcaaaatacttggtgtacctgaacgagacgatctttcaacgtcaggatattacccagaagaactttactcattggttatacaacactcagttgttaagtacatggattacggtgttcagcattcaatctataatgtgaactgtgaaccagtgaaattactacatgcccagttgggaatacgcatttctgattcaaaaccaatcgcaaagccagttaatcatttatacgacattgtcagttcgatttctccaatcagaatttctccaagtgatttaatcaaacaagctcgcgagtacaatctgacgtcgtataaagggaaaagggaatttttgttagatttaggtttaaatggtaacactctgaaaacatatttagcttccaaactactttttagagatcttatgctgtccaagtatgatgagctatactctactccaggattcggcgcgacacaattaacaacaatcccactggacatttcgtcagcggaaaaaatcttctctctaactcttaaattgccagctcacttgtatgaagtagtcatgctgctgttgttatatgaatatgtacatttcgtttttgcatgtaagaggacctttacggcgacactacatattacttcacaggatgacgctgccaaattaaccaaacagatactacaaatgttagatgacattaagttggataaggtttcatttaaagatgaagcctggtagatcatatccacataaaaacc

SEQ ID NO: 4 - protein, 1139 aa, unidentified
  VARIANT 32: note = X is any amino acid
  source 1..1139: note = Description of Unknown: Reoviridae family sequence; note = VP1 Pol
SLVYTNPKVAIVQFTRTDNEKLWQSKELNVLXPVELLTQLKNELDRAKTLDEKIEILLRLRYFTVYVEDKSDKRSIISSWLRKTITELGDEPEFESIQLIEHQARQWKIDNAGSLRAYHHNIPINEFIRDNEIEIIDTGDYKWKSDTLAGLNPNYYHRTHTLIGSVLFAIHSRLKFYTGDKKRALAYLLKVIEQCYSQGYLELSRNRKWSHTINELRKSNFRLYNAKVIHAACAMISLLHADPINAEFLCQIIAVYQIMPSHAAKVLSSPMTLYVGIATFPSRQVISTGNASECAPTSNPNNTFVAESQKNVWREAYKNDPLNHSKMLEIMNLNLTTNVGTFSLIFNCFSATFHVGHRVDNAQDAITEQVSVKYTSDVDKEMYDTYYYKLKQMLKDEIIQYVEEHIAKNYRDVTAESLSALANSSNGFIKEVEFVDRKIKTTKKILHLDSDLISNTYSDLTKSLSHGIPMGTRNVPARQTRGIFILPWQVAAIQHTIAESLYKRAKKGAYGGSFAEAYTAKTASLTYGVLAEDTSKAMRIILYTDVSQWDASQHNTVPYRSAWINAIREARSELRWNYSDEPYVLGMNVLDNMVKIQEYLLNSNLVVSSPGSLRPTKIIRYHGVASGEKTTKIGNSFANVALIETVLDYTKQQIPDLEISHLRVDGDDNVVTINTSCPIERLQAIIKNNYSKLNARVKALASYTGLEMAKRFVVCGKIFERGAIPIFTAERPYGTDLSTQSMCGSSIYSTAVNAYRGFGDEYFSFMLDVLVPPSSSVRVTGRLRVLLSPVTLYATGPLSFEVTPQGLGGRGRMFTESKKLFTLFKLLTQTVSVSVTPEDIKKYSATKQFNARTDVMIKSMRERVGGDAKALNRIMIDKEEQKTLGVPNVLSQKNRDQVSQAIKILGVPERDDLSTSGYYPEELYSLVIQHSVVKYMDYGVQHSIYNVNCEPVKLLHAQLGIRISDSKPIAKPVNHLYDIVSSISPIRISPSDLIKQAREYNLTSYKGKREFLLDLGLNGNTLKTYLASKLLFRDLMLSKYDELYSTPGFGATQLTTIPLDISSAEKIFSLTLKLPAHLYEVVMLLLLYEYVHFVFACKRTFTATLHITSQDDAAKLTKQILQMLDDIKLDKVSFKDEAW

SEQ ID NO: 5 - DNA, 2817 bp, unidentified
  source 1..2817: mol_type = other DNA; note = Description of Unknown: Reoviridae family sequence
attttcatgatggattcacagaatctaataaattctgcgaagaccaacattcaaagtgtagattcaaaggaagaaaaacaaaaaatctttgaccaattaacttctgatattagaagtcagaacgatgggaatattccagatgaggtgattgctgaattgcaacaactagcagagatcaatggtttaaaattttcgtttaagaaagaaggaaacaacgctgagatagtagaaccacctgatccaacttcagtcttatctcaagaggtatttcaaattaggacgattctatccaaaactttgtttgttgatgtggaactggaagattattccgtgtacgtaccaacagaaattaaccacttaacccccgtcctaatagatacgaagccgttacagacatatcaagttaaagcactaatgtacagagacaccgccatcataccatcaactagagatgatgtttcagatcaatacggagcggatgacattttatttgattcacatatgtttaatgatataaatcaagctcaaattcgcgacttcgatacgtacatccttgacaaagcggtcgcaattagatccacactacctaatctaaattttgtctccgcattggagaaagaagttaaccctttcaatgtgcataacactttatgtcttgacttcggtcaacgtgattattataaccttatatctgatagaaccaacctttcatttcagcaaagaagacaaagcatacaatttgacaacgttgtcgtggacggcgttgctcgtacggctagagtctctctccgtttgcatccacttgacagtcagctcctagatattgtaagattcaatacaatcgcggatcaacccttggctaatacactagctgaatatcaactgattgccgctgacggatttgttgctactccaaaatttagatttgacagagatatgcgtcttatagcagatgtacggtcattagttatggctagactgtgtgaattatcaccatactttcatcgaaccaggattctatcttcaatgatggattttaatcccctctggaaaaccaacgttttttcaagttcaattgataatgctaaagacgccatatacagaatggcagaaatttcatttactgtgtcagatgcgacaacatcagctctttcgactgtaaatgtagcatcagcacaacagaccttaatagtcttattaaatctttcacttttcaggtttgatatagatccaataggcagtcaatctaacttcggtgcagcagtttcagctgcattgatgttagttattttcccaacaaatgaagaagctatgtctaatgttacattcgacaatctatgcaacttggtgttcaatgaattaattgcatggacagttgagagaccaacttttgtgaaaagagcaggcgytacgaacgcgttcgaagccaatgtcaatattggaggaggcaacatgtctagagacatagtggcctacatgcgctatgttctcttacgtagaccgtgggctgtgtttcagaggtcttatgatgatggatatgtcgcagacattatgatcccaaacatagatgaggttaatgtcaatgaccaagcctatctagcgattaacaatcttttcaatggactgattcaatcggctcaacgcaatccaaatcctggcaggcagattgccgcaacatcatttagaaagcttctgaaatcgatgaaagattcatgttgcaacagaattatgccattaatcagattaatgagatacaacattgaaagagtagctagagtgtttaggcattttccatacacggcagacctcgtgaatagaataccagctttccgtgatgaaagattacgagttaaagtgccaatttctggagtgttatctataatgttgggaataaacaaggcaccagatttttttgactggtataatctgttaaaatttgctgacgttgtaagggcaaagaactttgctgagagagaatctttagaatcaataatggcccaagctgtaattagaaatgcaataaatccagcacgttcaaaaaaagattacatacaacaaaacgtcaagccggccactgccgttgtagcgtcgctcttgaagataccttcagctacgttcaccaccattctctccgataggatgttgaataacgaaatccgacgtactcaaagttttgacgtcataaaccgcataatggatgccgttcgcgcagcatttgaacatgtcccaacagccgaacatgggattgcaaagggcgctttacttctaccttatccacagaattttcagagatcatccgtatacgttaggaaagataacatcatttacaaccctccagttggaatagatagattcgcccttcacgatctactggacggtcgattctatcaaggaatggttaatagaattcagaatatggcgccttttgtgatagttggaccattacagacaaaatccgccgatggatctgctattgattctgttacgtctgcgtacttaactatgtcatctccatatgatgcatgcataagaccagaagatttacggcataataaaatagtacagccgccagtagttgattatttcagtgattcaagtattactagaccaaatacacaatttgaacagttgatgtcaaaaacatcagtattcattatcgacgccccaaaattgatagtgcaaagcgatgcaacggcatacacgtttgattataaagacattcaattgtccacatcagttacggacaaactggaatttacatcagtgcaaccaccagatgtcactttatttaatggaatgttggtctttgaggattag

SEQ ID NO: 6 - protein, 936 aa, unidentified
  VARIANT 468: note = X is any amino acid
  source 1..936: note = Description of Unknown: Reoviridae family sequence; note = VP2
MMDSQNLINSAKTNIQSVDSKEEKQKIFDQLTSDIRSQNDGNIPDEVIAELQQLAEINGLKFSFKKEGNNAEIVEPPDPTSVLSQEVFQIRTILSKTLFVDVELEDYSVYVPTEINHLTPVLIDTKPLQTYQVKALMYRDTAIIPSTRDDVSDQYGADDILFDSHMFNDINQAQIRDFDTYILDKAVAIRSTLPNLNFVSALEKEVNPFNVHNTLCLDFGQRDYYNLISDRTNLSFQQRRQSIQFDNVVVDGVARTARVSLRLHPLDSQLLDIVRFNTIADQPLANTLAEYQLIAADGFVATPKFRFDRDMRLIADVRSLVMARLCELSPYFHRTRILSSMMDFNPLWKTNVFSSSIDNAKDAIYRMAEISFTVSDATTSALSTVNVASAQQTLIVLLNLSLFRFDIDPIGSQSNFGAAVSAALMLVIFPTNEEAMSNVTFDNLCNLVFNELIAWTVERPTFVKRAGXTNAFEANVNIGGGNMSRDIVAYMRYVLLRRPWAVFQRSYDDGYVADIMIPNIDEVNVNDQAYLAINNLFNGLIQSAQRNPNPGRQIAATSFRKLLKSMKDSCCNRIMPLIRLMRYNIERVARVFRHFPYTADLVNRIPAFRDERLRVKVPISGVLSIMLGINKAPDFFDWYNLLKFADVVRAKNFAERESLESIMAQAVIRNAINPARSKKDYIQQNVKPATAVVASLLKIPSATFTTILSDRMLNNEIRRTQSFDVINRIMDAVRAAFEHVPTAEHGIAKGALLLPYPQNFQRSSVYVRKDNIIYNPPVGIDRFALHDLLDGRFYQGMVNRIQNMAPFVIVGPLQTKSADGSAIDSVTSAYLTMSSPYDACIRPEDLRHNKIVQPPVVDYFSDSSITRPNTQFEQLMSKTSVFIIDAPKLIVQSDATAYTFDYKDIQLSTSVTDKLEFTSVQPPDVTLFNGMLVFED

SEQ ID NO: 7 - DNA, 2339 bp, unidentified
  source 1..2339: mol_type = other DNA; note = Description of Unknown: Reoviridae family sequence
acattatgtcaaagctcattgagttttcagatctgggtgctgagatctcaaatagagaggaactttttaaactttcaaataacgtgtcatcttttgaggtaatcaaaccaacaaagaacattgaggattatattcgagacagtacacattatgtaatagtcgataggaggctaaataatgaagtttctgacatacttgatacactgtttcctacttcagttatttttaacagtgaggaaggctataagtttggtggttgtagacatcttcttgacaatgttcttcatgttagccaccatatgtacagctatcttaacgggagcgatcaaagttggctacctactgggtggtctgttagtgaatgcgatggatttgatgatcatattggagaccagataattaagcacatagtaaatacatgttcaatacagacagagggacaaatgaaaaatctaaaaccaggtacttatccaaaattagagagagttaatgaagctttcaaaggttttttaactaagattactgtacctcaaaccagtatggattttcagtcttataactatatggtgcagcgcaaacaaattggttatgtggtaagaaaaacagtgtttcatcttataagaaaacataactggaatgttaattacgtgggtcctgaatttgaatcttttaaagacatcattctactattaactgatcgcaactatactggtaaattcattacatacacactaaacactgctaagaaacacaactatagagcaaaagagagagaaaacaattctaaaaaagcaggatgggactttgtacataggctcagacataaatttgaaaatgcattttgtcatctaatgtacaatcatgttgcaaaacaacgtagctactcaacaatttatgttaacaagttgtataacgttggaaactgggtgggggcatattcatggttgaacataaacttagttgatcacttaccaaccattcagaaaaactcagtcgtgtttggatttctgttatcatctaaggattgttcattttccgtcaatactctatcggacattgtggtgtactcaccacaaccatatgatgatgactctaattactggactgtatcaataatgggagaatttataggacatttagcaaatgaagagtctagaatagcggaaaaaaacaacaacttaccaaactatgtatttggcggtgtcccatttacagctgaggcattggatcttaatcgaattacaatagcactttactcattgtccaatgcctttaattcacctgaactcatcaaagcaaccttatcatataatcatatatttacctttccaacgtactcagacggtaattggagagatgagagaccagtgcatgacaagatattcgttacgactcagaaacaactaaggtttgaagattggattattgacgtaaagaacttgtcattggagatgaacatagaggtggttgcagagagtgttttcctacaatttggacctcatagggcgttcataacggatatgtttcaacatatggtttcattcagattcaagcaagagaattttttttctgatcagaaaatgtcgcactttggaatccgtcagccttcaatacataacagagataagtatctttcatctaggctgaacgcctatatcaatagacaactaacactaggtacagatttaactgtgattagaaaaaacaacttcgcaggattttctgggcacttaatagcggtcgaaaaatactttcatgcactagtctacaccatgtctccgatgagatgggctaccagagctctttcagacgctacttacaaaaaacatgacaatttttctaatgctattggggaaaggcatacattggatgattttcgtaatacatatgcgtatcttggtagttccgttaatccaatactaaggtacaaacttgtcaacgataaatatgctgatgagccaaaatacgcaattatgttaatgtgtggaaataatcatatcactttacaactaactacaaaagatccatcaaatctcttaactgaaattggtaatataatacatggtttgaatatcaagtatttaggtaagaactcgtttggtaatatcaaactggccctataccagactaaagatatgttacaatataaactaagcaacttgctgagatcgtttgacataccttgtcagcagcatagaccatacataatgcacatgaccttgaaagatggcagcaacgtaccagatgtaataatagcacacagaagagatattaagatcaaagaaattaagcactgagctgacatactagaccagagtggtcggatacacataaaaacc

SEQ ID NO: 8 - protein, 763 aa, unidentified
  source 1..763: note = Description of Unknown: Reoviridae family sequence; note = VP3
MSKLIEFSDLGAEISNREELFKLSNNVSSFEVIKPTKNIEDYIRDSTHYVIVDRRLNNEVSDILDTLFPTSVIFNSEEGYKFGGCRHLLDNVLHVSHHMYSYLNGSDQSWLPTGWSVSECDGFDDHIGDQIIKHIVNTCSIQTEGQMKNLKPGTYPKLERVNEAFKGFLTKITVPQTSMDFQSYNYMVQRKQIGYVVRKTVFHLIRKHNWNVNYVGPEFESFKDIILLLTDRNYTGKFITYTLNTAKKHNYRAKERENNSKKAGWDFVHRLRHKFENAFCHLMYNHVAKQRSYSTIYVNKLYNVGNWVGAYSWLNINLVDHLPTIQKNSVVFGFLLSSKDCSFSVNTLSDIVVYSPQPYDDDSNYWTVSIMGEFIGHLANEESRIAEKNNNLPNYVFGGVPFTAEALDLNRITIALYSLSNAFNSPELIKATLSYNHIFTFPTYSDGNWRDERPVHDKIFVTTQKQLRFEDWIIDVKNLSLEMNIEVVAESVFLQFGPHRAFITDMFQHMVSFRFKQENFFSDQKMSHFGIRQPSIHNRDKYLSSRLNAYINRQLTLGTDLTVIRKNNFAGFSGHLIAVEKYFHALVYTMSPMRWATRALSDATYKKHDNFSNAIGERHTLDDFRNTYAYLGSSVNPILRYKLVNDKYADEPKYAIMLMCGNNHITLQLTTKDPSNLLTEIGNIIHGLNIKYLGKNSFGNIKLALYQTKDMLQYKLSNLLRSFDIPCQQHRPYIMHMTLKDGSNVPDVIIAHRRDIKIKEIKH

SEQ ID NO: 9 - DNA, 2287 bp, unidentified
  source 1..2287: mol_type = other DNA; note = Description of Unknown: Reoviridae family sequence
ccaggatgctttcttatctaagacgagaatggcagtcttatggggagagtgccacgaacgttaaagaggaggaagacacaacgaatgacaatggaaaaggaaataaagttgagaaaccagttaaagctgaaaacaggtattgttacagaagtccacaagaaaaagataaatatgaatcagatcttcaaggcttttcactgggaacgcaagatgaacatataaacccaacgacattacagatatacgatggaatattgagcaatggacatacgttcataaacacagacccaccttgctcgacaatcttcgaattgaatatcatggcagagagtggagcaatggttgataacaaaccactgatggatttcaattgttttataacatcaataaagaaagatgaaaacggggtatgtgatataacttatcactgcatgtcagacttggatgacgcgcagaaaagaatactactaagaggattctccagtaaaggttgttcaggattgaatgatatgaaagttacttctatacttcgcatagtggataaagctttagggtctgaatttcatacaagaactcaagcatcgttatatacgtgggatagtgactgcatcgaaagctatgatggtaaaatcagagttggtgaaagaaaagtaggcaactcgcgtataataatttacgagcaagaagatggtttctggaaaatattgaccgaaacactttggattgatttgagagctgtatttaaaccatatggaattatgggtggtgcttttaaaaattggctggtagattctggattcgacaaatatgaataccagtacagctacgaacgggaaggtaaaacggtttcggcaaccaccattacgtatccaaaaccaacaggaaaagccggcgtgaaccagccatggagaccggcaacagattacaatggtcagtatgtgtgtttacagccaggggatactttcacggtatggtattttgaagaccagtggcagattcgcaatgcgatatatgcaaaaaatttccaatccgacactatggcaaagggagtactggaaaacaaaggtccactaatttttaagatgaactatattccaagtctagccagtataaaaaataagccgggaaaagtgcaatacagatatatgaatggtggtttcgcgcaagttgacgcaagttcatatactggtatggcactgattttcaatttcgagtgtgttggtaaaaaattttacactgaagactataaaacgaaaattgacaattcaattacaccatatatatgttttattggaaaaaactacacacctgacgggtatttttatgaaaaaggatgttgttcagggtttgctgcaggatatgatacagaaacgatttcacacaaaatgacaatatcatatacggtgatgcgcccttcagatccggagttcgtaactggaggtgatgcatatggacaaagtatcacatcaaacctagaagtatctatacgagatcttcaagatcaaataaactcaatacgtgcagaactgaatatttcgcaagtaacgtcggcagtattttctgctatcacatctttaggtgatttaccgagtttattctcgaacataacacaaatatacagtaaactgaaagacgctcttttaaagcttaaaactagaaagagtaaaccaaaacctattaaagcaactatgatagtggatagaaacacgttagatgtaccaaatgtcagtatactaaatagaatgccagaagaattagaagtaggtatcatctacaattctataaggaaaccagaaaaacacgatattcctaaattcgccttgtctacagaattggaactaccttacatccaaactacatccactataactccaaaatttagaaaatatttagaagaaagagggctgctaacgatagatgatatagcggtacaatttgaccctttagatgtgacattttcaacacttaggagaaaaaatgctgaaatcatgaaatataagattgacccggaaatagcgyatgaagttctgtctcaaatgtctaactcagcaactagatcattgttttctctaaatgttaggaagcaaatatcaacacataatgagttttcaacaccaacatatgaacagctaataaatcgtattttgaatgataaagaaatattagatgtgttgggaaaactgaaccctcaatcagttggcaacatgttccaagagttcgttaacagaatgcaaaatatgttgtcttactattgagcatcctgaagcacgtatatattttaaaaacc

SEQ ID NO: 10 - protein, 749 aa, unidentified
  VARIANT 672: note = X is any amino acid
  source 1..749: note = Description of Unknown: Reoviridae family sequence; note = VP4
MLSYLRREWQSYGESATNVKEEEDTTNDNGKGNKVEKPVKAENRYCYRSPQEKDKYESDLQGFSLGTQDEHINPTTLQIYDGILSNGHTFINTDPPCSTIFELNIMAESGAMVDNKPLMDFNCFITSIKKDENGVCDITYHCMSDLDDAQKRILLRGFSSKGCSGLNDMKVTSILRIVDKALGSEFHTRTQASLYTWDSDCIESYDGKIRVGERKVGNSRIIIYEQEDGFWKILTETLWIDLRAVFKPYGIMGGAFKNWLVDSGFDKYEYQYSYEREGKTVSATTITYPKPTGKAGVNQPWRPATDYNGQYVCLQPGDTFTVWYFEDQWQIRNAIYAKNFQSDTMAKGVLENKGPLIFKMNYIPSLASIKNKPGKVQYRYMNGGFAQVDASSYTGMALIFNFECVGKKFYTEDYKTKIDNSITPYICFIGKNYTPDGYFYEKGCCSGFAAGYDTETISHKMTISYTVMRPSDPEFVTGGDAYGQSITSNLEVSIRDLQDQINSIRAELNISQVTSAVFSAITSLGDLPSLFSNITQIYSKLKDALLKLKTRKSKPKPIKATMIVDRNTLDVPNVSILNRMPEELEVGIIYNSIRKPEKHDIPKFALSTELELPYIQTTSTITPKFRKYLEERGLLTIDDIAVQFDPLDVTFSTLRRKNAEIMKYKIDPEIAXEVLSQMSNSATRSLFSLNVRKQISTHNEFSTPTYEQLINRILNDKEILDVLGKLNPQSVGNMFQEFVNRMQNMLSYY

SEQ ID NO: 11 - DNA, 1277 bp, unidentified
  source 1..1277: mol_type = other DNA; note = Description of Unknown: Reoviridae family sequence
ggtataatacgggtgtgctgtctgtacaagtcagggaaacctatgggaaataggcagtccagcttgcaatcgcagacgcatcgcactgatattaattctcataattcaaacattttcctccagagtgcatcttccgctgaatttagaactcagcatatactaattactgccggtgctgctttaatcgcctttctactcgctctccttatttcgagtctagtgtgtaattgttacctgctacgaaggttaagaaatggatctcgtaaaatttatcgcacaggcaaagtacaagaaggatcttatcccaatttatctaaacaattcattcgacccgaccactttgtctaaaaaacctttaaagttagaactcattaagtataacaatccatttcgtgaggtgcctcaactcgcaggggaatctcttttactggaagatgtttgcccttacaaccatgagcatttctgtggcggcatacacattccaaagcaattcaacatgtcaccaaaaggaagaattagtcatatcacaaatgacaaaattgcatgrccatgcggtttggaatctataattattgatggaaagaaatacactggttccgcatttgtcaaatgtagatgcgggcaaccatatccaacaattattgaccaaccatcgactttcttcttcctaacttgttgttcaaatgatacgaagtcagtgaaaattggattaagtgaaaacttcaattgtttaaattgtaacagaagtgtgcgttggttcagtccaggaagaggactgaggactacgcatcaatattaccttccaagtcagctatgtccagcatgcatgccatttcgcgatttagtcgcttcaatgtctttattaaacaaagtggaatttcttggcgctgactttgctcaaatgcgcactgattttgagtggaaaagacagttgcagaacaactgtgaatctgcctttagatcattcaactctccacaattagcgcatagagttacaccatacgaaaggattgatccgaatctcactctcaacactgttactgagaccatttcctcagtgaacagaagacagcacaacaaaatagaaatcactccaatatgcagaggaaagatcgcgattagcgacggatacagacgatgcattcttgattttcaagataatgctgaatttcactgctcagttaatatgctattgcttcgctggaagctcatctgaacatagaatgagagttccagatctctaacctgacgcagccactcaccctatccgaaaaacc

SEQ ID NO: 12 - protein, 320 aa, unidentified
  source 1..320: note = Description of Unknown: Reoviridae family sequence; note = NSP1
  VARIANT 98: note = X is any amino acid
MDLVKFIAQAKYKKDLIPIYLNNSFDPTTLSKKPLKLELIKYNNPFREVPQLAGESLLLEDVCPYNHEHFCGGIHIPKQFNMSPKGRISHITNDKIAXPCGLESIIIDGKKYTGSAFVKCRCGQPYPTIIDQPSTFFFLTCCSNDTKSVKIGLSENFNCLNCNRSVRWFSPGRGLRTTHQYYLPSQLCPACMPFRDLVASMSLLNKVEFLGADFAQMRTDFEWKRQLQNNCESAFRSFNSPQLAHRVTPYERIDPNLTLNTVTETISSVNRRQHNKIEITPICRGKIAISDGYRRCILDFQDNAEFHCSVNMLLLRWKLI

SEQ ID NO: 13 - DNA, 1259 bp, unidentified
  source 1..1259: mol_type = other DNA; note = Description of Unknown: Reoviridae family sequence
ggattaaataacccaactgacgcttcaagcatggatctgattgaaacagttaatgcatgcgttagattgcaaaaaagaattctaaacttagctccgaacacgaatttgaacacatcaggccaaagcactctgaatgactacaatgcattggcttcaaggtgcaatgggagaacatacgcttttcttgatcaaaccgcagttcttactccttttaccattaacgccccaatcatttctctctcggtccgtatatccacagatgattatgatgacatgaggtctggaataaactcaattttagacgtgctcgccgctgctattagaacagaaggatctagaccagtaagagcaatagaaagacgtgttcttgaacccgccgttaaacagttggtagaagacctaaagctgaaaagtctaacttccgaaatttccgttgcgaatttagcagctgtagatactgccataattcaaccagaaatcattcaaactgagaatccattatatgccgatgttattgaacaagttgtaaacaggccacaaatgaatatgactggcggaaatattagwgctactttaggaaggtggtcaggcaacaaaggagtagttacatgtatgtcaggaatggattcggaacacagatttactgttgaccttaaagctaggaccactggaataatcaatatcgtatatgtgccaaccgctggtacgatactagtaccaatgccgaatggaaggaatagagaaggtaaactaattgatgtctcagcagaaatgatggctgagaattttgccattgatttcatggatgatgatgcaatacttcagacagaaactggagttggagtctattcctttccaatgtgtaaccgcattagattcaggatctctccatgggatatgcaaaaagatcaagatggtctaggcacagttcatatgacgaactgggcacaggggacggctgccaggcaaccagctatctctttcatgttcgaaactcgtgatacctttactcaaggtgactaccaacatttatccaagtgcacacccaagtcacaatatgtgttggacacagttttccctgaaacatcattcgtaaacaaaccaaacatcgactggaacattcagagcctacttacttccactattcaaccagtatggtgtaagaaaatagcaattttagtgtctgcatacgctgcgaaaatctgatccggcgtcagcctccaaaggaggtgcttcgagaggtgtgaaaaaagaataca

SEQ ID NO: 14 - protein, 391 aa, unidentified
  VARIANT 179: note = X is any amino acid
  source 1..391: note = Description of Unknown: Reoviridae family sequence; note = VP6
MDLIETVNACVRLQKRILNLAPNTNLNTSGQSTLNDYNALASRCNGRTYAFLDQTAVLTPFTINAPIISLSVRISTDDYDDMRSGINSILDVLAAAIRTEGSRPVRAIERRVLEPAVKQLVEDLKLKSLTSEISVANLAAVDTAIIQPEIIQTENPLYADVIEQVVNRPQMNMTGGNIXATLGRWSGNKGVVTCMSGMDSEHRFTVDLKARTTGIINIVYVPTAGTILVPMPNGRNREGKLIDVSAEMMAENFAIDFMDDDAILQTETGVGVYSFPMCNRIRFRISPWDMQKDQDGLGTVHMTNWAQGTAARQPAISFMFETRDTFTQGDYQHLSKCTPKSQYVLDTVFPETSFVNKPNIDWNIQSLLTSTIQPVWCKKIAILVSAYAAKI

SEQ ID NO: 15 - DNA, 972 bp, unidentified
  source 1..972: mol_type = other DNA; note = Description of Unknown: Reoviridae family sequence
tattgtgtttgattcacatagctcgctgagagtcaagcacaatggccttgaacgtgatcgtgtctgttctgaagaacgtattgaatgagttgagtgttgagaagtctgatgtgataactgataaattcaaggaggctttaaacgactgtggcattagagtcgatgattggagagatgctttctacaacgagagaataccaaagccaatgtcttccacctcaatggcgatgcaactgaagaactttgaacttgaggtactacagcttagaaacagagcttggttagagggagctgataggaaaaacagattgttttcgagttttgatatctccggcaaaaatggacatacagttcttataccgaaaacaagaaatgctcagatgttgttggctaactctgtggccgacttaaaaatcagaaatggccagtctgaagtcattgatgatctaattaagaagaatgaggagctaaagagacaattggaccaatttacagtagctgtagagaatccaagagaacttcagataacttatcaggttgaggcagctaccgcaaaaatgacggaactgtctgatctgcttaaggtgtgccaaaatgaatgcgttaaacttcaaaaaagactgacgttccaagaggaacagactgatgatagaattgccgcaatcaatatgcatcatgctgaagaaacaaaaattcttaaaagggaaattgaacgacwwaacatagtaaatgcatgtgtgaaggattacaacgatgtgttggaaaaacagcatgaaagaaatctaaaaataattcgaggactagctttaaaggctggtttactggttgaagattcagacgatgaagtagaagtggacagcatgaaacaagaaaaatgaatgattgatatgaacggaatttaaacttcatgacatagacgcgtcgcacgtttaaggataaaaagttggactgaccacccaatcactaaaaacc

SEQ ID NO: 16 - protein, 278 aa, unidentified
  VARIANT 226: note = X is any amino acid
  source 1..278: note = Description of Unknown: Reoviridae family sequence; note = Nsp3
MALNVIVSVLKNVLNELSVEKSDVITDKFKEALNDCGIRVDDWRDAFYNERIPKPMSSTSMAMQLKNFELEVLQLRNRAWLEGADRKNRLFSSFDISGKNGHTVLIPKTRNAQMLLANSVADLKIRNGQSEVIDDLIKKNEELKRQLDQFTVAVENPRELQITYQVEAATAKMTELSDLLKVCQNECVKLQKRLTFQEEQTDDRIAAINMHHAEETKILKREIERXNIVNACVKDYNDVLEKQHERNLKIIRGLALKAGLLVEDSDDEVEVDSMKQEK

SEQ ID NO: 17 - DNA, 1030 bp, unidentified
  source 1..1030: mol_type = other DNA; note = Description of Unknown: Reoviridae family sequence
ggttttaaaatagttggtgtagtgtgtcgtgagagggctccatcaccctggtcaccatgacgcagtctatttctcttgctgactttgttacaaaaactgatgacggtttcatgccatctgatcgtgagtgcactgttttggatcgatacttgtcaaaagatcagaaagaattacgcgaaacgtacaaggatgaaaagaatcatagagcatcactcagagtcaagatgtttctaacttctgcaccatcacgcaggttcacacagcaaggtgtcgtgccaatgcgtgaattaagaacgaactcagacataccgagctcacttttaaatattatcactgattggttgatgaacgttctgagcgatgaggaaaatcaggagatgtttgaagaatttattagctcaaaatttccagatattcttggatcagctgataagctggctagatttgctttgagactggagaacaaaagagacataatacacaaaaacttttcgaaagccatgaatgcttttggaacatgttttctcgccgtcaaaccaacttatgcaacagaaggtcagtgtaatgttgtacgtgccacggacgatgcaatcattttagagtttcaaccaattccagaatattatagatgtggcaggtcaagatctaccttctataagttatacccgctttcagaagagcaaccagttacaggaatgattgcattaaggggaattgcagggaatcaacttataatgaatcatggacatgggcatctaagaaccgtaccataccatgaaatagctgatgcaatcaaatcatttgcaaagaaggacaaagaaacattagaactgatctccaaatcacctctttcagcacagtgcggaagcaaatttttggacatgcttgacggtatcagatcgaagcagagaattgaggatgtgattgccaaagcgaagacttttgaaaagaaaaaaccatgaatatggagtcaaaagataaatatgacygtgataaatccgctacatcggctattaacatataaaaaccc

SEQ ID NO: 18 - protein, 301 aa, unidentified
  source 1..301: note = Description of Unknown: Reoviridae family sequence; note = Nsp2
MTQSISLADFVTKTDDGFMPSDRECTVLDRYLSKDQKELRETYKDEKNHRASLRVKMFLTSAPSRRFTQQGVVPMRELRTNSDIPSSLLNIITDWLMNVLSDEENQEMFEEFISSKFPDILGSADKLARFALRLENKRDIIHKNFSKAMNAFGTCFLAVKPTYATEGQCNVVRATDDAIILEFQPIPEYYRCGRSRSTFYKLYPLSEEQPVTGMIALRGIAGNQLIMNHGHGHLRTVPYHEIADAIKSFAKKDKETLELISKSPLSAQCGSKFLDMLDGIRSKQRIEDVIAKAKTFEKKKP

SEQ ID NO: 19 - DNA, 745 bp, unidentified
  source 1..745: mol_type = other DNA; note = Description of Unknown: Reoviridae family sequence
ggtattatagagtcttaggatggctgaggacggcgaaatggaacaaatgataatagatactctgtataatcaaattcttaatttagctggaaatacgacgcatgaaaacatcaaacaaactatttcaaactcttcgccgcaaaagattttaactggcgcatttttaacattaggaacattgttaacaacaattgtatttaagagaaagggaatcaatctacttacgagtaaatttaagtccaacattacgtatttagctgaaatattggtatggaaggctgagcagacagtaaatgagataattgagaagacattacagcaacacaagtttatgagggaaaaagatattgatggtgttttgaaggacattaagaaaatgaaatatgacatagaacatattggtggattagacgtaacgaaagaacttttttcaatgtgtgaacgcaaaatgatcgatttagatgataaaatgagagacatagaaaaatcttgtgatagaagaatacgggattacgactggaaaataaacgcactaacactacatccgatacagcaagctgcggtgcagagcgaaacagcagaaaagaacgacaaaattgaaatcgtagatgaaacagtacaaaacacacgcagtaagcctattaaaactcgtctggctcccaaaagactctaacgacttgtggaataattaggagaagatctctctgtggttattcctccccatcaagtcaagcgacataaaacc

SEQ ID NO: 20 - protein, 217 aa, unidentified
  source 1..217: note = Description of Unknown: Reoviridae family sequence; note = Nsp4
MAEDGEMEQMIIDTLYNQILNLAGNTTHENIKQTISNSSPQKILTGAFLTLGTLLTTIVFKRKGINLLTSKFKSNITYLAEILVWKAEQTVNEIIEKTLQQHKFMREKDIDGVLKDIKKMKYDIEHIGGLDVTKELFSMCERKMIDLDDKMRDIEKSCDRRIRDYDWKINALTLHPIQQAAVQSETAEKNDKIEIVDETVQNTRSKPIKTRLAPKRL

SEQ ID NO: 21 - DNA, 583 bp, unidentified
  source 1..583: mol_type = other DNA; note = Description of Unknown: Reoviridae family sequence
ggtatataaaagtcagtaaacggctggaaacgttgaactgactactcattgcccaggaatggctgaagcatctgagttcaatttcaatacaagaaggaaaagaagaacaattactgagaaacgtgagactaaagaggttgttaaacagaagatgcttgttgacgagaaaaatgataattacgaagaagttgagtcagcttcagtatattctgcggaatcatccagaagcaactatagtgatgcatatgagaaacttaaacgtgaaccagtggttgaagattcaaatgatgaaaaatataaaactttagaatactcagaagacgaagaagttttcaaatctgcattgaaacaaagtgacaaaccagctcgaagcatcagcaaaacacagcatgatgacagtactgattctagtgttctcatggaaaaaatatccgaactttctttggaaattgaaaagatgaaacagtctaatcaaccattyacagttgatgcagcattcaatactacactcagaaatgttgacaaccttactacaagacagaaacaggcgctgattatggcactcgtaaattcaatgaactaa

SEQ ID NO: 22 - protein, 174 aa, unidentified
  VARIANT 141: note = X is any amino acid
  source 1..174: note = Nsp5; note = Description of Unknown: Reoviridae family sequence
MAEASEFNFNTRRKRRTITEKRETKEVVKQKMLVDEKNDNYEEVESASVYSAESSRSNYSDAYEKLKREPVVEDSNDEKYKTLEYSEDEEVFKSALKQSDKPARSISKTQHDDSTDSSVLMEKISELSLEIEKMKQSNQPXTVDAAFNTTLRNVDNLTTRQKQALIMALVNSMN

SEQ ID NO: 23 - DNA, 25 bp, synthetic construct
  source 1..25: mol_type = other DNA; note = RotaB Forward Primer
cagacgatctgatagggatgtattg

SEQ ID NO: 24 - DNA, 22 bp, synthetic construct
  source 1..22: mol_type = other DNA; note = RotaB Reverse Primer
atgtccgtgacgtagtatcttc

SEQ ID NO: 25 - DNA, 23 bp, synthetic construct
  source 1..23: mol_type = other DNA; note = Astrovirus Forward Primer
gtgcagatgtgttggcgtataag

SEQ ID NO: 26 - DNA, 23 bp, synthetic construct
  source 1..23: mol_type = other DNA; note = Astrovirus Reverse Primer
tgaagcgtacaaaccaggatgag

(19) United States
(12) Patent Application Publication
(10) Pub. No.: US 2025/0011730 A1
(43) Pub. Date: Jan. 9, 2025

(54) NOVEL PORCINE ROTAVIRUS

(71) Applicant: INTERVET INC., Madison, NJ (US)

(72) Inventors: Ad DE GROOF, Groesbeek (NL); Paul VERMEIJ, St. Anthonis (NL); Cornelia Maria VAN DER HOEK, Amsterdam (NL); Martin DEIJS, Huizen (NL)

(73) Assignee: INTERVET INC., Madison, NJ (US)

(21) Appl. No.: 18/742,851

(22) Filed: Jun. 13, 2024

Related U.S. Application Data
(63) Continuation of application No. 17/291,769, filed on May 6, 2021.

(30) Foreign Application Priority Data
Nov. 12, 2018 (EP) 18205672.1

(51) Int. Cl. C12N 7/00; C07K 14/005

(52) U.S. Cl. CPC C12N 7/00; C07K 14/005; C12N 2720/12321; C12N 2720/12322; C12N 2720/12334

(57) ABSTRACT

The present invention pertains to a novel rotavirus, especially an isolated virus, which is a member of the sub-species of porcine group B rotavirus (porcine RVB), causing diarrheal disease in pigs, to DNA fragments and corresponding proteins of the said virus, to vaccines on the basis of said virus, DNA and/or protein and to antibodies reactive with said virus and/or protein and diagnostic test kits for the detection of said virus.

### CROSS-REFERENCE TO RELATED APPLICATIONS

This application is a continuation of U.S. patent application Ser. No. 17/291,769, file May 6, 2021, which is a U.S. National Stage Application under 35 U.S.C. § 371 of International Patent Application No. PCT/EP2019/080811, filed Nov. 11, 2019, which claims priority to European Patent Application No. 18205672.1, filed November 12. 2018, the disclosure of each of which is incorporated by reference herein in its entirety.

### SEQUENCE LISTING

This application contains an electronic Sequence Listing which has been submitted in XML format via Patent Center, the entire content of which is incorporated by reference herein in its entirety. The Sequence Listing XML file submitted via Patent Center is entitled “14463-332-999_SEQ_LISTING.xml”, was created on Jun. 12, 2024, and is 56,113 bytes in size.

The present invention pertains to a novel rotavirus causing diarrheal disease in pigs, to nucleic acid fragments and corresponding proteins of the said virus, to vaccines on the basis of said virus, nucleic acid and/or protein and to antibodies reactive with said virus and/or protein and diagnostic test kits for the detection of said virus.

### GENERAL BACKGROUND

Over the last decades, world-wide a strong increase is seen in the consumption of pig meat. As a consequence, an increase is seen in the number and the size of farms, in order to meet the increasing needs of the market. As is known from animal husbandry in general, large numbers of animals living closely together are vulnerable to all kinds of diseases. Moreover, farming of large numbers of animals increases the danger of infection with such diseases. One of these diseases that especially occur in young animals, such as pigs, is an infection with rotavirus.

Rotavirus (RV) is well established as a major cause of acute gastroenteritis in young children and animals, including nursing and weaned piglets. Rotaviral enteritis is a mild to severe disease characterized by nausea, vomiting, watery diarrhea and low-grade fever. Once a subject is infected by the virus, there is an incubation period of about two days before symptoms appear. The period of illness is acute. Symptoms often start with vomiting followed by four to eight days of profuse diarrhea. Dehydration is more common in rotavirus infection than in most of those caused by bacterial pathogens, and is the most common cause of death related to rotavirus infection. Further, these rotaviruses are a potential reservoir for genetic exchange with human rotaviruses. As a pathogen of livestock, notably in young calves and piglets, rotaviruses cause economic loss to farmers because of costs of treatment associated with high morbidity and mortality rates.

RVs belong to the Reoviridae family, possess a genome composed of 11 segments of double-stranded RNA (dsRNA) and are currently classified into eight groups (A-H) based on antigenic properties and sequence-based classification of the inner viral capsid protein 6 (VP6). While human RVA and RVC have been described around the world. current reports indicate that human RVB strains have been described only in China. Porcine RVB were first identified in the 1980s.

Serological and molecular characterization of RVB strains is limited due to the difficulty of adapting RVB strains to cell culture. Classification of porcine RVB strains based on pairwise identities of the genes encoding the outer capsid protein VP7 has been proposed by Kuga et al., 2009 (Arch Virol. 2009; 154 (11): 1785-95) and Marthaler et al., 2012 (Virology. 2012 Nov. 10; 433 (1): 85-96). Kuga et al. sequenced the VP7 of 38 porcine RVB strains and constructed phylogenetic trees and pairwise identity frequency graphs for G genotype classification purposes. Based on their analyses. they proposed 5 genotypes which were further divided into 12 clusters, using 67% and 76% nucleotide cut-off values. Marthaler et al. developed an adapted VP7 classification (in the following the “Marthaler classification”) using previously published and newly sequenced RVB strains. resulting in 20 G genotypes based on an 80% nucleotide identity cut off value.

Rotavirus outer capsid proteins VP7 and VP4 have been well established to be capable of inducing independent neutralizing antibodies (Greenberg et al., I. Virol., 1983, 47: 267-275; Hoshino et al., Proc. Natl. Acad. Sci. USA. 1985, 82:8701-8704) and associated protection against disease. While VP4 is located on the surface of the virion that protrudes as a spike. VP7 is a glycoprotein that forms the outer surface of the virion. Apart from its structural functions, it determines the G-genotype of the strain and, along with VP4, is involved in immunity to infection.

Recently, a neonatal diarrhea outbreak was observed in piglets in a farm in Spain. although the sows were vaccinated against RVA. Thus, maternal antibodies against RVA should have been present in the colostrum due to vaccination. Thus, samples were collected from infected subjects and analyzed for the presence of viruses.

A nucleic acid sequence belonging to a rotavirus B VP7 protein was found. which can be linked to the presence of RVB in the samples. The presence of RVB in the samples could also explain the clinical observations. The almost full-length nucleotide sequence encoding the VP7 protein is presented in SEQ ID NO: 1.

It was surprisingly found by genetic analyses that the VP7 sequence detected in the samples was genetically distinct from VP7 sequences from known RVB genotypes. Thus, it was concluded that the subjects suffered from a previously unknown disease causing RVB type.

### OBJECT OF THE INVENTION

It is thus an objective of the present invention to provide a new infectious agent associated with diarrhea in pigs as well as vaccines aiming at protecting, i.e. preventing, ameliorating and/or treating, a pig against the disease or at least reducing symptoms of the disease and/or decreasing the mortality of the disease. Moreover, it is an objective of the present invention to provide means to detect and identify the disease-associated infectious agent.

### SUMMARY OF THE INVENTION

The present invention provides an isolated rotavirus which is a member of the sub-species of porcine group B rotaviruses genotype G12, which in its wild type form causes (neonatal) diarrhea in pigs, said virus being characterized in that it has a viral genome comprising an open reading frame having a nucleotide sequence corresponding to the nucleotide sequence depicted in SEQ ID NO: 1or a nucleotide sequence having a level of identity of at least 90% therewith. Although the present virus is in fact an RNA virus, it is common to express that an open reading frame of the viral genome has a level of identity corresponding to a particular DNA sequence. As is commonly known, this expresses that the actual RNA sequence of the viral genome can be transcribed from that DNA sequence.

Further, the present invention provides a nucleic acid fragment (either DNA or RNA) comprising an open reading frame comprising at least 100 nucleotides, characterized in that said nucleic acid fragment has a level of identity of at least 90% to the nucleotide sequence depicted in SEQ ID NO: 1, as well as a protein encoded by the nucleic acid fragment. Preferably the fragment comprises more than 100 nucleotides, such as 120, 140, 160, 180, 200, 220, 240, 260, 280, 300, 320, 340, 360, 380, 400, 420, 440, 460, 480, 500, 520, 540, 560, 580, 600, 620, 640, 660, 680, 700, 720, 730 up to 740 nucleotides.

The present invention further provides an outer viral capsid glycoprotein VP7, characterized that it is encoded by a nucleic acid fragment having a level of identity of at least 90% to the nucleotide sequence depicted in SEQ ID NO: 1. An example of such a protein is depicted in SEQ ID NO: 2.

The present invention further provides a vaccine for protecting a pig against an infection caused by porcine RVB, characterized in that said vaccine comprises an immunogenically effective amount of a virus as described herein and a pharmaceutically acceptable carrier.

The present invention further provides a vaccine for protecting a pig against an infection caused by porcine RVB, characterized in that said vaccine comprises an immunologically effective amount of a protein or an outer viral capsid glycoprotein VP7 or a nucleic acid fragment as described herein, and a pharmaceutically acceptable carrier.

The vaccine may be used in prophylactically treating an animal.

The present invention further provides an antibody or antiserum reactive with a virus or with a protein or with an outer viral capsid glycoprotein VP7 as described herein.

The present invention further provides a diagnostic test kit for the detection of antibodies reactive with a virus or with antigenic material thereof or reactive with a protein or reactive with an outer viral capsid glycoprotein VP7 as described herein.

The present invention further provides a diagnostic test kit for the detection of a virus or antigenic material thereof or an outer viral capsid glycoprotein VP7 as described herein.

### DETAILED DESCRIPTION OF THE INVENTION

It has been found that the causative agent of the disease symptoms described above may be linked to the presence of a novel rotavirus B (RVB), in particular a disease causing RVB of the G12 genotype. Although RVB of genotype G12 has been isolated from diseased pigs infected with various rotaviruses (sec Marthaler 2012), this is the first time a RVB of genotype 12 is unambiguously associated with disease in pigs. Genetic analyses revealed that the nucleotide sequence found in the samples belong to an open reading frame of an outer viral capsid protein VP7 (in short: “VP7 capsid protein” or “VP7”) of RVB. The nucleotide sequence of the DNA fragment is depicted in SEQ ID NO: 1. The corresponding amino acid sequence is depicted in SEQ ID NO: 2.

The maximum identity of the almost full-length DNA fragment of SEQ ID NO: 1 with known DNA sequences is 87% identity with a porcine VP7 gene classified as genotype G12 included in the classification proposal by Marthaler et al., 2012. The VP7 classification proposed by Marthaler et al. resulted in 20 G genotypes based on an 80% nucleotide identity cut off value. However, Marthaler et al. also reported an overlap between inter-and intra-genotype identities. Next to this, none of the G12 genotype RotaB viruses has ever been unambiguously associated with disease in pigs, let alone with neonatal diarrhea.

For this reason, the inventors decided to tentatively place the nucleotide sequence of SEQ ID NO: 1 as belonging to a novel virus, especially a novel porcine RVB, in particular an RVB of a novel genotype, the virus containing a VP7 encoding gene with highest similarities to the G12 genotype within the Marthaler classification.

A phylogenetic tree showing the relation between the VP7 DNA sequence of the novel virus according to the invention with known VP7 sequences of RVB is provided in FIG. 1, consisting of two subfigures, the first one denoted as “Figure B”, the second one denoted as “Figure A”. The phylogenetic tree was created with MEGA-X software using the neighbor-joining method, with pairwise deletion in case of gaps or insertions. Reference: Tamura K, et al., MEGA6: molecular evolutionary genetics analysis using maximum likelihood, evolutionary distance, and maximum parsimony methods. Mol. Biol. Evol. 2011:28: 2731-2739. The new virus is referred to in this tree (first part, lower section) as New RotaB VP7 Spain.

Thus, in one embodiment the present invention provides the VP7 protein encoded by the DNA fragment of SEQ ID NO: 1 for use as an antigen in a method for protecting pigs against a pathogenic infection with porcine RVB, and in particular against a pathogenic infection with porcine RVB of the G12 genotype. The VP7 protein is typically comprised in a vaccine composition, i.e. a composition safe to be administered to pigs, and in which VP7 is mixed with a pharmaceutically acceptable carrier, as will be described below.

It will be understood that for these genes and proteins natural variations can exist between individual representatives of rotavirus. Genetic variations leading to minor changes in e.g. the capsid protein sequences, such as VP7, do exist. First of all, there is the so-called “wobble in the second and third base” explaining that nucleotide changes may occur that remain unnoticed in the amino acid sequence they encode: e.g. triplets TTA, TTG, TCA, TCT, TCG and TCC all encode Leucine. In addition, minor variations between representatives of the novel virus according to the invention may be seen in amino acid sequence. These variations can be reflected by (an) amino acid difference(s) in the overall sequence or by deletions, substitutions, insertions, inversions or additions of (an) amino acid(s) in said sequence. Amino acid substitutions which do not essentially alter biological and immunological activities, have been described, e.g. by Neurath et al. in “The Proteins” Academic Press New York (1979). Amino acid replacements between related amino acids or replacements which have occurred frequently in evolution are, inter alia, Ser/Ala, Ser/Gly, Asp/Gly, Asp/Asn, Ile/Val (see Dayhof, M.D., Atlas of protein sequence and structure, Nat. Biomed. Res. Found., Washington D.C., 1978, vol. 5, suppl. 3). Other amino acid substitutions include Asp/Glu, Thr/Ser, Ala/Gly, Ala/Thr, Ser/Asn, Ala/Val, Thr/Phe, Ala/Pro, Lys/Arg, Leu/Ile, Leu/Val and Ala/Glu. Based on this information, Lipman and Pearson developed a method for rapid and sensitive protein comparison (Science 227, 1435-1441, 1985) and determining the functional similarity between homologous proteins. Such amino acid substitutions of the exemplary embodiments of this invention, as well as variations having deletions and/or insertions arc within the scope of the invention.

This explains why the VP7 protein, when isolated from different representatives of a porcine rotavirus according to the invention, may have homology levels that are significantly below 100%, while still representing the VP7 protein of the porcine rotavirus according to the invention.

This is clearly reflected e.g. in the phylogenetic tree in FIG. 3 of the paper by Marthaler et al., 2009 cited above, where it is shown that even within one single clade the VP7 encoding nucleotide sequences nevertheless have significantly different overall genomic nucleotide sequences.

Thus, the virus according to the invention is described i.a. as an isolated virus being characterized in that it its genome comprises a nucleotide sequence that corresponds to a nucleotide sequence having a level of identity of at least 90% to the nucleotide sequence depicted in SEQ ID NO: 1. In particular, the virus according to the invention is characterized in that it is a rotavirus, which is a member of the sub-species of porcine group B rotavirus (porcine RVB), and wherein the nucleotide sequence depicted in SEQ ID NO: 1 belongs to an open reading frame encoding a VP7.

For the purpose of this invention, a “level of identity” is to be understood as the level of identity of the sequence of SEQ ID NO: 1 and/or the corresponding region encoding the VP7 of a porcine rotavirus and/or the corresponding amino acid sequence of which the level of identity has to be determined. A suitable program for the determination of a level of identity is the nucleotide blast program (blastn) of NCBFs Basic Local Alignment Search Tool, using the “Align two or more sequences” option and standard settings (http://blast.ncbi.nlm.nih.gov/Blast.cgi). Therein, the identities are based on the (standard) Blastn algorithm that is used in BLAST (Stephen Altschul, Warren Gish, Webb Miller, Eugene Myers, and David J. Lipmann at National Institutes of Health (NIH), Journal of Molecular Biology, 1990).

The virus according to the invention is typically an isolated virus. For the purpose of this invention, “isolated” means: set free from tissue with which the virus is associated in nature.

A preferred form of this embodiment relates to a virus, such as an isolated virus, that comprises a nucleotide sequence that has a level of identity of at least 91%, more preferably at least 92%, 93%, 94%, 95%, 96%, 97%, 98%, 99% or even 100%, in that order of preference, to the nucleotide sequence as depicted in SEQ ID NO: 1.

The virus according to the invention can be in a live, a live attenuated or an inactivated form. As indicated above, the DNA sequence of the gene encoding the VP7 of the virus is characterized. The identification of the nucleotide sequence of SEQ ID NO: 1 as belonging to an ORF encoding a VP7 is highly useful, since it can now be used i.a. as a basis for DNA or RNA vaccines, for use in the preparation of subunit vaccines on the basis of the encoded protein, or for diagnostic purposes, as will extensively be explained below.

Therefore, in another embodiment, the present invention relates to a nucleic acid fragment (e.g. DNA or RNA) comprising an open reading frame comprising at least 100, preferably at least 200, more preferably at least 300, 400 or 500 nucleotides, characterized in that said nucleic fragment has a level of identity of at least 90% to the nucleotide sequence depicted in SEQ ID NO: 1, preferably at least 91%, more preferably at least 92%, 93%, 94%, 95%, 96%, 97%, 98%, 99% or even 100%, in that order of preference, to the nucleotide sequence depicted in SEQ ID NO: 1.

In still another embodiment, the present invention relates to a nucleic acid fragment as described above, characterized in that the open reading frame encodes a VP7 or a fragment thereof.

The term “fragment thereof” as used in the present invention may relate to any smaller part of the full-length VP7 protein. For use in vaccination, such parts should be suitable to impart immunogenic properties, i.e. the fragment is functional in inducing an immune response in the vaccinated subject, and this can be designated as “immunogenic fragment”.

The nucleic acid fragment according to the present invention may be brought into a form suitable for heterologous expression of the encoded protein. Such a form may be a vector or other form suitable for heterologous expression of viral nucleic acid. Typically, the vector contains a promotor suitable for heterologous expression. Thus, in a preferred embodiment, the nucleic acid fragment according to the present invention is under the control of a heterologous promoter or is introduced into an open reading frame under the control of a heterologous promoter.

Therefore, in another embodiment, the present invention relates to a protein, in particular an outer viral capsid glycoprotein VP7 encoded by the nucleic acid fragment according to the present invention, or a fragment thereof, produced in a heterologous expression system. A preferred form of this embodiment relates to a VP7 having the amino acid sequence as depicted in SEQ ID NO: 2.

Such VP7 of the virus according to the invention are suitable for use as an antigen, in particular for use as an antigen in vaccines, more specifically in subunit vaccines. Further, they can be used to raise antibodies. Furthermore, they make diagnostic tests possible, as explained below.

Further, the present invention provides the nucleotide and amino acid sequences of the entire coding regions of the different proteins of the novel virus, confirming that the novel virus belongs to a rotavirus. The nucleotide and amino acid sequences provided by the present invention are given by the SEQ ID NO. 1-22 as follows:

| Segment, | | | |
|---|---|---|---|
| Nr | Type | Protein | Sequence, Nr. |
| 1 | Nucleotide | | SEQ ID NO: 3 |
| 1 | Amino acid | VP1 Pol | SEQ ID NO: 4 |
| 2 | Nucleotide | | SEQ ID NO: 5 |
| 2 | Amino acid | VP2 | SEQ ID NO: 6 |
| 3 | Nucleotide | | SEQ ID NO: 7 |
| 3 | Amino acid | VP3 | SEQ ID NO: 8 |
| 4 | Nucleotide | | SEQ ID NO: 9 |
| 4 | Amino acid | VP4 (VP8*, VP5*) | SEQ ID NO: 10 |
| 5 | Nucleotide | | SEQ ID NO: 11 |
| 5 | Amino acid | NSp1 | SEQ ID NO: 12 |
| 6 | Nucleotide | | SEQ ID NO: 13 |
| 6 | Amino acid | VP6 | SEQ ID NO: 14 |
| 7 | Nucleotide | | SEQ ID NO: 15 |
| 7 | Amino acid | NSp3 | SEQ ID NO: 16 |
| 8 | Nucleotide | | SEQ ID NO: 17 |
| 8 | Amino acid | NSp2 | SEQ ID NO: 18 |
| 9 | Nucleotide | | SEQ ID NO: 1 |
| 9 | Amino acid | VP7 | SEQ ID NO: 2 |
| 10 | Nucleotide | | SEQ ID NO: 19 |
| 10 | Amino acid | NSp4 | SEQ ID NO: 20 |
| 11 | Nucleotide | | SEQ ID NO: 21 |
| 11 | Amino acid | NSp5 | SEQ ID NO: 22 |

*VP4 is an open reading frame that encodes a preprotein that is subsequently cleaved into VP5 and VP8

Thus, the virus according to the invention may further be described as an isolated virus being characterized in that it its genome comprises a nucleotide sequences that correspond to a nucleotide sequences each having a level of identity of at least 90% to the nucleotide sequences depicted above encoding the proteins of Segments 1-11, i.e. to the nucleotide sequences of SEQ ID NO: 1, 3, 5, 7, 9, 11, 13, 15, 17, 19 and 21.

It is one of the merits of the present invention that it is now for the first time possible to analyze the presence or absence of the novel rotavirus in the various organs and body fluids of pigs suffering from viral diarrhea, or other signs and symptoms associated with rotavirus infection, and to treat the disease or prevent outbreak of the disease in healthy animals by administering to the animal a vaccine as described in the following.

A “vaccine” according to the present invention is a pharmaceutical composition that is safe to administer to a subject animal, and is able to induce protective immunity in that animal against a pathogenic micro-organism, i.e. to induce protection against the micro-organism.

“Protection” against a micro-organism means aiding in preventing, ameliorating and/or treating (including curing) a pathogenic infection with that micro-organism or a disorder arising from that infection, for example to prevent or reduce one or more clinical signs resulting from the infection with the pathogen.

Thus, another embodiment of the present invention relates to a vaccine for protecting, i.e. preventing, ameliorating and/or treating, against a rotavirus infection in pigs, in particular infections caused by porcine RVB, further particularly infections caused by porcine RVB of the G12 genotype, wherein such vaccines comprise a virus according to the invention and a pharmaceutically acceptable carrier. Protecting in this respect should thus be interpreted to comprise vaccination in order to prevent the outbreak of the disease, i.e. (neonatal) diarrhea or gastroenteritis caused by viral infection, as well as vaccination to diminish the symptoms of the disease.

Preferably, the vaccine according to the invention is used for preventing the disease given the short time between infection and outbreak of the disease of about two days and given that viral diarrhea is highly contagious.

In another embodiment, the present invention relates to a vaccine for protecting against neonatal diarrhea caused by infection with porcine RVB. Therefore, the present invention is further directed to a vaccine for sow vaccination.

“Sow vaccinations” in the present invention means prepartum immunization of sows to convey passive immunity to piglets and provide protection against an infection with the porcine RVB as described herein. By inducing antibodies in the female animal, piglets arrive at adequate protection against the infection through the intake of colostrum of the vaccinated animal. Therefore, an antigen that is shown to have a protective effect in piglets, can be useful for vaccinating sows to arrive at a clear protective effect in piglets, typically at least in the window between day (and day 21 after birth.

The present invention thus also pertains to the use of a vaccine as described herein for vaccination of a female pig and allowing the piglet to take up colostrum form the vaccinated female pig. To arrive at optimum protection, the colostrum is typically taken up within 48 hours, in particular within 24 hours after birth of the piglet.

Examples of pharmaceutically acceptable carriers that are suitable for use in a vaccine according to the invention are, for example, sterile water, saline, and aqueous buffers such as PBS. In addition, a vaccine according to the invention may comprise other additives such as adjuvants, stabilizers, anti-oxidants and others, as described below.

A vaccine according to the invention may comprise the virus according to the invention in attenuated live or inactivated form.

Attenuated live virus vaccines, i.e. vaccines comprising the virus according to the invention in a live attenuated form, have the advantage over inactivated vaccines that they best mimic the natural way of infection. In addition, their replicating abilities allow vaccination with low amounts of viruses; their number will automatically increase until it reaches the trigger level of the immune system. From that moment on, the immune system will be triggered and will finally eliminate the viruses. A live attenuated virus is a virus that has a decreased level of virulence when compared to virus isolated from the field. A virus having a decreased level of virulence is considered a virus that does not cause the typical symptoms of viral infection. A possible disadvantage of the use of live attenuated viruses however might be that inherently there is a certain level of virulence left. This is not a real disadvantage as long as the level of virulence is acceptable, i.e. as long as the vaccine at least prevents the pigs from suffering from diarrhea or other typical symptoms of the infection. Of course, the lower the rest virulence of the live attenuated vaccine is, the less influence the vaccination has on weight gain during/after vaccination. Therefore, one preferred form of this embodiment of the invention relates to a vaccine comprising a virus according to the invention wherein said virus is in a live attenuated form.

Attenuated viruses can e.g. be obtained by growing the viruses according to the invention in the presence of a mutagenic agent, followed by selection of virus that shows a decrease in progeny level and/or in replication speed. Many such agents are known in the art.

Inactivated vaccines are, in contrast to their live attenuated counterparts, inherently safe, because there is no rest virulence left. In spite of the fact that they usually comprise a somewhat higher dose of viruses compared to live attenuated vaccines, they may e.g. be the preferred form of vaccine in pigs that are suffering already from other diseases. Pigs that are kept under sub-optimal conditions, such as incomplete nutrition or sub-optimal housing would also benefit from inactivated vaccines.

Therefore, another preferred form of this embodiment relates to a vaccine comprising a virus according to the invention wherein said virus is in an inactivated form.

The standard way of inactivation is a classical treatment with formaldehyde. Other methods well-known in the art for inactivation are UV-radiation, gamma-radiation, treatment with binary ethylene-imine, and thimerosal. The skilled person knows how to apply these methods. Preferably the virus according to the invention is inactivated with β-propiolactone, glutaraldehyde, ethylene-imine or formaldehyde. It goes without saying that other ways of inactivating the virus are also embodied in the present invention.

Although whole inactivated rotavirus provides a good basis for vaccines, their production may be expensive and laborious. In particular, adapting porcine RVB strains to cell culture is still found difficult in the art (Sanekata et al., J Clin Microbiol. 1996 March; 34 (3): 759-761).

An alternative approach is to develop subunit or recombinant vaccines by expressing one or more rotavirus proteins or immunogenic parts thereof which retain the neutralizing epitopes necessary for effective recognition by the host cell. Both VP7 and VP4, the two protein components of the outer capsid, react with neutralizing antibodies, and monoclonal antibodies (MAbs) directed at either of these proteins are capable of neutralizing rotavirus.

VP7 is the major outer capsid protein and is primarily responsible for determining the viral antigenic characteristics. It is a highly immunogenic glycoprotein and it is thus a primary candidate for inclusion in a subunit vaccine. Hence, an attractive alternative for the use of whole viruses is the use of rotavirus subunits, more preferably the use of the subunit formed by VP7. Subunits formed by VP7 have the advantage that they do not have to be inactivated before use in a vaccine, and therefore they have the additional advantage that they are intrinsically safe. Further, cloning and heterologous expression systems for the heterologous production of such subunits are available and established in the art, as described below. Further, it has been shown that recombinant VP7 mediates native antigenic determinants in the absence of other rotavirus proteins (Khodabandchloo et al., Iran J Public Health. 2012; 41 (5): 73-84.).

Recombinant vaccines can also be provided as nucleic acid construct containing vaccines such as DNA and RNA vaccines, including synthetic messenger RNA, RNA replicons, and naked DNA vectors. One such vaccination strategy includes the use of alphavirus-derived replicon RNA particles (RP) [Vander Veen, et al. Anim Health Res Rev. 13 (1): 1-9. (2012) doi: 10.1017/S1466252312000011; Kamrud et al., J Gen Virol. 91 (Pt 7): 1723-1727 (2010)] which have been developed from several different alphaviruses, including Venezuelan equine encephalitis virus 25 (VEE) [Pushko et al., Virology 239:389-401 (1997)], Sindbis (SIN) [Bredenbeck et al., Journal of Virology 67:6439-6446 (1993)], and Semliki Forest virus (SFV) [Liljestrom and Garoff, Biotechnology (NY) 9:1356-1361 (1991)]. RP vaccines deliver propagation-defective alphavirus RNA replicons into host cells and result in the expression of the desired antigenic transgene(s) in vivo [Pushko et al., Virology 30 239 (2): 389-401 (1997)]. RPs have an attractive safety and efficacy profile when compared to some traditional vaccine formulations [Vander Veen, et al. Anim Health 6 Res Rev. 13 (1): 1-9. (2012)]. The RP platform has been used to encode pathogenic antigens and is the basis for several USDA-licensed vaccines for swine and poultry.

Thus, another embodiment of the present invention relates to vaccines for protecting, i.e. preventing, ameliorating or treating, a pig against a rotavirus infection, in particular infections caused by porcine RVB, further particularly infections caused by porcine RVB of the G12 genotype, wherein such vaccines comprise an immunogenically effective amount of the VP7 protein or immunogenic fragments thereof, or corresponding nucleic acid constructs, and a pharmaceutically acceptable carrier.

Expression of rotavirus VP7 has been reported in the art for E. coli, herpes virus, vaccinia virus in mammalian cells and baculovirus. However, most of them were not full-length VP7 protein. Advanced technique in anchoring the simian rotavirus SA11 VP7 to the surface of eukaryotic cells (VP7sc) has done using recombinant vaccinia virus and adenoviruses. The expressed VP7 protein appeared to be both antigenic and immunogenic and induced passive protection against rotavirus disease in mice (Both et al., Virology. 1993; 193:940-950).

Using the right system for viral gene expression is very important in producing biologically active recombinant protein. Baculovirus expression system has some unique features that made it the system of choice for many protein expressions, such as solubility, correctly folding, signal peptide cleavage, oligomerization, functional activity, phosphorylation, and glycosylation of recombinant proteins. Baculovirus has been used successfully in the art as an expression system for the production of rotavirus proteins (McGonigal TP et al., Virus Res. 1992: 23 (1-2): 135-150; Redmont MJ et al., Vaccine. 1993; 11:273-281.; Fiore L et al., J Gen Virol. 1995;76 (Pt 8): 1981-1988). The baculovirus system is thus a candidate for the expression of VP7 in that it offers the possibility of synthesis of a recombinant protein in high yield with the conformational requirements necessary to permit immunological and functional studies (Fiore L et al., J Gen Virol. 1995;76 (Pt 8): 1981-1988; Ishida SI et al. J clin Microbiol. 1996;34 (7): 1694-1700).

By far most expression systems currently in use for making rotavirus capsid proteins are baculovirus-based expression systems. Methods for the production of highly immunogenic rotavirus capsid proteins in baculovirus-based expression systems have been e.g. described in the art (McGonigal TP et al., Virus Res. 1992; 23 (1-2): 135-150)

Furthermore, baculovirus expression systems and baculovirus expression vectors in general have been described extensively in textbooks (Baculovirus Expression Vectors, A Laboratory Manual. By David R. O'Reilly, Lois K. Miller, and Verne A. Luckow. Publisher: Oxford University Press, USA, May 1994; and Baculovirus and Insect Cell Expression Protocols. In: Methods in Molecular Biology™, Volume 388 (2007). Editors: David W. Murhammer).

Baculovirus-based expression systems are also commercially available, e.g. from Invitrogen Corporation, 1600 Faraday Avenue, Carlsbad, California 92008, USA. An alternative for Baculovirus-based expression systems are yeast-based expression systems. Yeast expression systems are e.g. described in: Production of recombinant proteins: novel microbial and eukaryotic expression systems by Gerd Gellissen, ISBN: 3-527-31036-3. Ready-to-use expression systems are i.a. commercially available from Research Corp. Technologies, 5210 East Williams Circle, Suite 240, Tucson, AZ 85711-4410 USA. Yeast and insect cell expression systems are also e.g. commercially available from Clontech Laboratories, Inc. 4030 Fabian Way, Palo Alto, California 94303-4607, USA. Alternatively, recombinant expression can be performed by using the Alpha Vax® Alphavaccine Platform System technology using viral replicon particles (RP) based on a modified alphavirus.

Therefore, VP7 or fragments thereof can be obtained by heterologous expression of an ORF comprising the gene encoding the VP7 or a fragment thereof in a suitable expression system, such as a baculovirus expression system. Recombinant VP7 or fragments thereof can readily be made in large amounts and they are highly immunogenic.

Expression of recombinant VP7 or fragments thereof is of course also possible in mammalian cell-based expression systems as known in the art, but these systems would most likely be more expensive to use, when compared to the baculovirus-based expression systems.

The amount of recombinant VP7 in a vaccine is typically about 1 to 10 times, preferably 2 to 5 times, the amount used for whole virus vaccine. For example, Khodabandehloo et al., Iran J Public Health. 2012; 41 (5): 73-84 reported an antibody titer for recombinant VP7, which is about four times lower compared to antibodies against the whole virus, which is explained with the additional presence of the immunogenic VP4 in the whole virus. Usually, an amount of between 1 and 100 μg of the recombinant VP7 would be very suitable as a vaccine dose. An amount of up to 500 μg could become necessary in case of using immunogenic fragments of VP7, wherein the amount necessary to achieve immunization depends on the length of the recombinant protein. From a point of view of costs, a preferred amount would be in the range of 1-50 μg of recombinant VP7, more preferred in the range of 1-25 μg. The route of administration would be comparable with that of inactivated whole virus particles.

A vaccine according to the invention on the basis of inactivated whole virus or recombinant VP7 or an immunogenic fragment thereof preferably comprises an adjuvant. Conventional adjuvants, well-known in the art are e.g. Freund's Complete and Incomplete adjuvant, vitamin E, non-ionic block polymers, muramyl dipeptides, Quill AR, mineral oil, e.g. Bayol® or Markol®, vegetable oil, and Carbopol® (a homopolymer), or Diluvac® Forte. The vaccine may also comprise a so-called “vehicle”. A vehicle is a compound to which the polypeptide adheres, without being covalently bound to it. Often used vehicle compounds are e.g. aluminum hydroxide, -phosphate or -oxide, silica, Kaolin, and Bentonite.

In principle a vaccine according to the invention can be given just once. However, especially in the case of inactivated vaccines, be it whole virus vaccines, recombinant VP7 or immunogenic fragments, preferably a first and maybe even a second booster vaccination is given. A first booster would usually be given at least two weeks after the first vaccination. A very suitable moment for a booster vaccination is between 3 and 16 weeks after the first vaccination. A second booster, if necessary, would usually be given between 4 and 50 weeks after the first booster.

An alternative to the inactivated whole virus vaccine approach and the recombinant VP7 approach is the use of live recombinant non-rotavirus vectors that have pigs as their host animal, as carriers of the novel porcine RVB VP7 gene or an immunogenic fragment thereof.

Amongst the suitable recombinant non-rotavirus vectors that have pigs as their host animal, two vectors are especially suitable as carriers: Pseudorabies virus (PRV) and Classical Swine Fever Virus (CSFV). The use of such recombinant viruses in vaccines has the additional advantage that the vaccinated animals become at the same time vaccinated against both PRV and RVB or CSFV and RVB.

Live attenuated CSFV vectors are also very suitable as live recombinant vectors. Merely as an example; live attenuated CSFV from which the N<pro> gene has been deleted, has been described by Mayer et al. (Immunochemical Methods in Cell and Molecular Biology, Academic Press, London, 1987). Such a live attenuated virus allows, i.a. at the site of the deletion of the N<pro> gene, for the insertion of the VP7 gene. Such a live recombinant CSFV vector equally forms a suitable carrier for the VP7 gene of the present invention.

The expression of the VP7 gene or a fragment thereof according to the invention can be brought under the control of any suitable heterologous promoter that is functional in a mammalian cell (see below).

A heterologous promoter is a promoter that is not the promoter responsible for the transcription of the VP7 gene in the wild-type form of the novel porcine rotavirus according to the invention. It may be a rotavirus promoter responsible for the transcription of VP7 of another rotavirus, that does not belong to the virus according to the invention or it may be a non-rotavirus promoter.

Therefore, another embodiment of the present invention relates to a DNA fragment comprising a gene encoding a VP7 or a fragment thereof according to the invention, characterized in that said gene is under the control of a functional heterologous promoter. A promoter that is functional in a mammalian cell is a promoter that is capable of driving the transcription of a gene that is located downstream of the promoter in a mammalian cell.

Examples of suitable promoters that are functional in a mammalian cell include classic promoters such as the (human) cytomegalovirus immediate early promoter (Seed, B. et al., Nature 329, 840-842, 1987; Fynan, E.F. et al., PNAS 90, 11478-11482,1993; Ulmer, J.B. et al., Science 259, 1745-1748, 1993), Rous sarcoma virus LTR (RSV, Gorman, CM. et al., PNAS 79, 6777-6781, 1982; Fynan et al., supra; Ulmer et al., supra), the MPSV I.TR (Stacey et al., J. Virology 50, 725-732, 1984), SV40 immediate early promoter (Sprague J. et al., J. Virology 45, 773, 1983), the SV-40 promoter (Berman, P.W. et al., Science, 222, 524-527, 1983), the metallothionein promoter (Brinster, R.L. et al., Nature 296, 39-42, 1982), the heat shock promoter (Voellmy et al., Proc. Natl. Acad. Sci. USA, 82, 4949-53, 1985), the major late promoter of Ad2 and the β-actin promoter (Tang et al., Nature 356, 152-154, 1992). The regulatory sequences may also include terminator and poly-adenylation sequences. Amongst the sequences that can be used are the well-known bovine growth hormone poly-adenylation sequence, the SV40 poly-adenylation sequence, the human cytomegalovirus (hCMV) terminator and poly-adenylation sequences.

Thus, another form of this embodiment relates to a vaccine for protecting a pig against an infection caused by RVB, characterized in that said vaccine comprises a live recombinant non-RVB vector comprising a DNA fragment comprising a gene encoding a VP7 or an immunogenic fragment thereof according to the invention under the control of a functional promoter and a pharmaceutically acceptable carrier.

Further, the live recombinant non-RVB vector should be expressing an immunogenically effective amount of VP7 or immunogenic fragment thereof.

An alternative for vaccination with an inactivated whole virus vaccine, a recombinant VP7 vaccine or a live recombinant non-RVB vector, is the use of DNA vaccination.

Such DNA vaccination is based upon the introduction of a DNA fragment carrying the gene encoding the VP7 according to the invention under the control of a suitable promoter, into the host animal. Once the DNA is taken up by the host's cells, the gene encoding the VP7 or fragment thereof is transcribed and the transcript is translated into VP7 in the host's cells. This closely mimics the natural infection process of the rotavirus. Suitable promoters are promoters that are functional in mammalian cells, as exemplified above.

A DNA fragment carrying the gene encoding the VP7 or fragment thereof under the control of a suitable promoter could e.g. be a plasmid. This plasmid may be in a circular or linear form.

Examples of successful DNA vaccination of pigs are i.a. the successful vaccination against Aujeszky's disease as described in Gerdts et al., Journal of General Virology 78:2139-2146 (1997). They describe a DNA vaccine wherein a DNA fragment is used that carries glycoprotein C under the control of the major immediate early promoter of human cytomegalovirus. Vaccination was done four times with two weeks intervals with an amount of 50 μg of DNA. Vaccinated animals developed serum antibodies that recognized the respective antigen in an immunoblot and that exhibited neutralizing activity.

Another example of successful DNA vaccination of pigs is given by Gorres et al., Clinical Vaccine Immunology 18:1987-1995 (2011). They described successful DNA vaccination of pigs against both pandemic and classical swine H1N1 influenza. They vaccinated with a prime vaccination and 2 homologous boosts at 3 and 6 weeks post priming, of a DNA vaccine comprising the HA gene of influenza H1N1 under the control of a functional promoter. Therefore, again another form of this embodiment relates to a vaccine for protecting a pig against an infection caused by RVB, characterized in that said vaccine comprises a DNA fragment comprising a VP7 gene or fragment thereof according to the present invention under the control of a functional promoter, and a pharmaceutically acceptable carrier.

Further, the DNA fragment comprising a gene encoding VP7 or fragment thereof according to the invention should be expressing an immunogenically effective amount of VP7 or immunogenic fragment thereof.

What constitutes an “immunogenically effective amount” of a vaccine according to the invention that is based upon a whole virus according to the invention, recombinant VP7 according to the invention, or immunogenic fragment thereof, a live recombinant vector or a DNA vaccine according to the invention depends on the desired effect and on the target organism. The term “immunogenically effective amount” as used herein relates to the amount of virus, recombinant protein, live recombinant vector or DNA vaccine that is necessary to induce an immune response in pigs to the extent that it decreases the pathological effects caused by infection with a wild-type rotavirus, when compared to the pathological effects caused by infection with a wild-type rotavirus in non-immunized pigs.

The determination whether a treatment is “immunologically effective”, can be achieved, for instance, by administering an experimental challenge infection to vaccinated animals and next determining a target animal's clinical signs of disease, serological parameters or by measuring re-isolation of the pathogen, followed by comparison of these findings with those observed in field-infected pigs.

The amount of virus administered will depend on the route of administration, the presence of an adjuvant and the moment of administration.

A preferred amount of a live vaccine comprising virus according to the invention is expressed for instance as Tissue Culture Infectious Dose (TCID50). For instance, for a live virus a dose range between 10 and 10⁹ TCID50 per animal dose may advantageously be used, depending on the rest virulence of the virus. Preferably a range between 10² and 10⁶ TCID50 is used.

Many ways of administration can be applied, all known in the art. Vaccines according to the invention are preferably administered to the animal via injection (intramuscular or via the intraperitoneal route) or per os.

The protocol for the administration can be optimized in accordance with standard vaccination practice. In all cases, administration through an intradermal injector (IDAL) is one way of administration. If a vaccine comprises inactivated virus or recombinant protein according to the invention, the dose would also be expressed as the number of virus particles to be administered. The dose would usually be somewhat higher when compared to the administration of live virus particles, because live virus particles replicate to a certain extent in the target animal, before they are removed by the immune system. For vaccines on the basis of inactivated virus, an amount of virus particles in the range of about 10⁴ to 10⁹ particles would usually be suitable. The amount may depend on the adjuvant used, but is typically within the defined range.

If a vaccine comprises recombinant protein according to the invention, the dose could also be expressed in micrograms of protein. For vaccines on the basis of recombinant protein, a suitable dose would usually be in the range between 1 and 500 micrograms of protein. The does, again, may depend on the adjuvant used.

If a vaccine comprises a DNA fragment comprising a gene encoding VP7, the dose would be expressed in micrograms of DNA. A suitable dose would usually be in the range between 5 and 500 micrograms of DNA. The dose may depend, i.a., on the efficiency of the expression plasmid used. Typically, an amount of between 20 and 50 micrograms of plasmid per animal would be sufficient for an effective vaccination.

A vaccine according to the invention may take any form that is suitable for administration in the context of pig farming, and that matches the desired route of application and desired effect. Preparation of a vaccine according to the invention is carried out by means conventional for the skilled person.

For oral administration the vaccine is preferably mixed with a suitable carrier for oral administration i.e. cellulose, food or a metabolisable substance such as alpha-cellulose or different oils of vegetable or animal origin.

In practice, swine are vaccinated against a number of pathogenic viruses or micro-organisms.

Therefore, it is highly attractive, both for practical and economic reasons, to combine a vaccine according to the invention for pigs with e.g. an additional immunogen of a virus or micro-organism pathogenic to pigs, or genetic information encoding an immunogen of said virus or micro-organism.

Thus, a preferred form of this embodiment relates to a vaccine according to the invention, wherein that vaccine comprises at least one other pig-pathogenic microorganism or pig-pathogenic virus and/or at least one other immunogenic component and/or genetic material encoding said other immunogenic component, of said pig-pathogenic microorganism or pig-pathogenic virus. An immunogen or immunogenic component is a compound that induces an immune response in an animal. It can e.g. be a whole virus or bacterium, or a protein or a sugar moiety of that virus or bacterium.

The most common pathogenic viruses and micro-organisms that are pathogenic for swine are Brachyspira hyodysenteriae, African Swine Fever virus, Nipah virus, Porcine Circovirus, Porcine

Torque Teno virus, Pseudorabies virus, Porcine influenza virus, Porcine parvovirus, Porcine respiratory and Reproductive syndrome virus (PRRS), Porcine Epidemic Diarrhea virus (PEDV), Foot and Mouth disease virus, Transmissible gastro-enteritis virus, Rotavirus, Escherichia coli, Erysipelo rhusiopathiae, Bordetella bronchiseptica, Salmonella cholerasuis, Haemophilus parasuis, Pasteurella multocida, Streptococcus suis, Mycoplasma hyopneumoniae and Actinobacillus pleuropneumoniae.

Therefore, a more preferred form of the invention relates to a vaccine according to the invention, wherein the virus or micro-organism pathogenic to swine is selected from the group of Brachyspira hyodysenteriae, African Swine Fever virus, Nipah virus, Porcine Circovirus, Porcine Torque Teno virus, Pseudorabies virus, Porcine influenza virus, Porcine parvovirus, Porcine respiratory and Reproductive syndrome virus (PRRS), Porcine Epidemic Diarrhea virus (PEDV), Foot and Mouth disease virus, Transmissible gastro-enteritis virus, Rotavirus, Escherichia coli, Erysipelo rhusiopathiae, Bordetella bronchiseptica, Salmonella cholerasuis, Haemophilus parasuis, Pasteurella multocida, Streptococcus suis, Mycoplasma hyopneumoniae and Actinobacillus pleuropneumoniae.

Still another embodiment relates to a method for the preparation of a vaccine according to the invention, wherein the method comprises the mixing of a virus according to the invention and/or a recombinant protein according to the invention and/or a DNA fragment encoding a VP7 or a fragment thereof according to the invention and/or a live recombinant non-rotavirus vector encoding a VP7 or a fragment thereof according to the invention, and a pharmaceutically acceptable carrier.

In another embodiment, the present invention relates to a virus according to the invention and/or a recombinant protein and/or a VP7 or an immunogenic fragment thereof according to the invention and/or a DNA fragment encoding a VP7 or an immunogenic fragment thereof according to the invention and/or a live recombinant non-rotavirus vector encoding a VP7 or an immunogenic fragment thereof according to the invention, for use in a vaccine.

In another embodiment, the present invention relates to a method for the preparation of a vaccine as defined herein, characterized in that said method comprises the mixing of a virus, or an immunogenetically effective amount of a VP7 or of an immunogenic fragment thereof or a recombinant vector encoding the VP7 or the immunogenic fragment thereof, as defined herein, and a pharmaceutically acceptable carrier.

In another embodiment, the present invention relates to a virus, a DNA fragment, a VP7 or an immunogenic fragment thereof, or a recombinant vector encoding the VP7 or the immunogenic fragment thereof, as defined herein, for use in the manufacture of a vaccine for protecting a pig against an infection caused by porcine RVB.

As mentioned above, rotavirus infection is highly contagious. This means that it is important to know if rotavirus is present in a certain pig-population well before the first clinical signs become manifest. Thus, for efficient protection against disease, a quick and correct detection of the presence of rotavirus is important.

Therefore, it is another objective of this invention to provide diagnostic tools suitable for the detection of an infection of a virus according to the invention.

These tools partially rely on the availability of antibodies against the virus. Such antibodies can e.g. be used in diagnostic tests for rotavirus infection. Antibodies or antiserum comprising antibodies against the virus according to the invention can quickly and easily be obtained through vaccination of e.g. pigs, poultry or e.g. rabbits with the virus according to the invention followed, after about four weeks, by bleeding, centrifugation of the coagulated blood and decanting of the sera. Such methods are well-known in the art.

Other methods for the preparation of antibodies raised against the virus according to the invention, which may be polyclonal, monospecific or monoclonal (or derivatives thereof) are also well-known in the art. If polyclonal antibodies are desired, techniques for producing and processing polyclonal sera are well-known in the art for decades. Monoclonal antibodies, reactive against the virus according to the invention can be prepared by immunizing inbred mice by techniques also long known in the art.

Thus, another embodiment of the present invention relates to antibodies or antisera that are reactive with the virus according to the invention.

A diagnostic test kit based upon the detection of a virus according to the invention or antigenic material of that virus and therefore suitable for the detection of RVB infection may e.g. comprise a standard ELISA test. In one example of such a test the walls of the wells of an ELISA plate are coated with antibodies directed against the virus. After incubation with the material to be tested, labeled antibodies reactive with the virus are added to the wells. If the material to be tested would indeed comprise the novel virus according to the invention, this virus would bind to the antibodies coated to the wells of the ELISA. Labeled antibodies reactive with the virus that would subsequently be added to the wells would in turn bind to the virus and a color reaction would then reveal the presence of antigenic material of the virus.

Therefore, still another embodiment of the present invention relates to diagnostic test kits for the detection of a virus according to the invention or antigenic material of the virus, that comprise antibodies reactive with a virus according to the invention or with antigenic material thereof. Antigenic material of the virus is to be interpreted in a broad sense. It can be e.g. the virus in a disintegrated form, or viral envelope material comprising viral outer membrane proteins. As long as the material of the virus reacts with antiserum raised against the virus, the material is considered to be antigenic material.

A diagnostic test kit based upon the detection in serum of antibodies reactive with the virus according to the invention or antigenic material of the virus and therefore suitable for the detection of RVB infection may also e.g. comprise a standard ELISA test. In such a test the walls of the wells of an ELISA plate can e.g. be coated with the virus according to the invention or antigenic material thereof. After incubation with the material to be tested, e.g. serum of an animal suspected from being infected with the novel virus according to the invention, labeled antibodies reactive with the virus according to the invention are added to the wells. If antibodies against the novel virus according to the invention would be present in the tested serum, these antibodies will bind to the viruses coated to the wells of the ELISA. As a consequence, the later added labeled antibodies reactive with the virus would not bind and no color reaction would be found. A lack of color reaction would thus reveal the presence of antibodies reactive with the virus according to the invention.

Therefore, still another embodiment of the present invention relates to diagnostic test kits for the detection of antibodies reactive with the virus according to the invention or with antigenic material of the virus that comprise the virus according to the invention or antigenic material thereof.

The design of the immunoassay may vary. For example, the immunoassay may be based upon competition or direct reaction. Furthermore, protocols may use solid supports or may use cellular material. The detection of the antibody-antigen complex may involve the use of labeled antibodies; the labels may be, for example, enzymes, fluorescent-, chemoluminescent-, radio-active- or dye molecules.

Suitable methods for the detection of antibodies reactive with a virus according to the present invention in the sample include, in addition to the ELISA mentioned above, immunofluorescence test (IFT) and Western blot analysis.

An alternative but quick and easy diagnostic test for diagnosing the presence or absence of a virus according to the invention is a PCR test as referred to above, comprising a PCR primer set reactive with a specific region of the VP7 DNA fragment of the novel virus according to the invention. Specific in this context means unique for e.g. the VP7 gene of the novel virus, i.e. not present in other members of the family of Reoviridae.

By simple computer-analysis of the novel VP7 gene sequence provided by the present invention with the known VP7 gene of other rotaviruses, the skilled person is able to develop specific PCR-primers for diagnostic tests for the detection of the novel virus and/or the discrimination between the novel virus and other viral (porcine) pathogens.

Thus, another embodiment relates to a diagnostic test kit for the detection of a virus according to the invention, characterized in that said test kit comprises a PCR primer set that is specifically reactive with a region of the VP7 gene sequence of the DNA fragment of the virus according to the invention.

### EXAMPLES

### Example 1: Identification of a New Rotavirus in Piglets

Recently, a neonatal diarrhea outbreak with clear clinical signs of rotavirus infection was observed in piglets in a farm of a large pork producer in Spain, although the sows were vaccinated against RVA.

Clinical symptoms observed in infected pigs were diarrhea at first week of live, with yellow feces of different consistency, sometimes liquid and sometimes thick feces. Piglets of primiparous and multiparous sows were affected. Morbidity was very high (40% of the litters in some batches), mortality 5-6%.

Due to diarrheal symptoms, pigs were vaccinated using Coliclos Prosystem® RCE (Merck Animal Health), a vaccine for use in swine as an aid in the prevention of rotaviral diarrhea, enterotoxemia and colibacillosis in nursing piglets, the vaccine containing two major Rotavirus A genotypes (G4, G5), four major E. coli pilus antigens and C. perfringens type C toxoid. Vaccination, however, did not result in disappearance of symptoms.

In particular, involvement of a virus was suspected because “feedback” of infected material resulted in immunity and disappearance of symptoms.

Maternal antibodies against RVA should have been present in the colostrum due to vaccination. Samples were sent for laboratory analysis, without a plausible explanation of the rotavirus signs despite the vaccination regime.

Samples were collected by taking rectal swabs, from subjects including 6 groups of sows with having clear rota clinical signs (the “rota groups”), and of 3 groups having no signs (“control groups”. From each group, rectal swabs were taken from the sow and three piglets. The samples were analyzed for the presence of viruses by VIDISCA (Virus discovery based on cDNA-AFLP (amplified fragment length polymorphism), a method originally described by van der Hoek et al., (Nat Med. 2004; 10:368-373). Virus discovery based on VIDISCA is a novel approach that provides a fast and effective tool for amplification of unknown genomes, e.g., of human pathogenic viruses. The VIDISCA method is based on double restriction enzyme processing of a target sequence and ligation of oligonucleotide adaptors that subsequently serve as priming sites for amplification. As the method is based on the common presence of restriction sites, it results in the generation of reproducible, species-specific amplification patterns. The method allows amplification and identification of viral RNA/DNA, with a lower cutoff value of 10 (5) copies/ml for DNA viruses and 10 (6) copies/ml for the RNA viruses.

Using the VIDISCA method, the nucleotide sequence of SEQ ID No. 1 and having the amino acid sequence of SEQ ID No. 2 belonging to a VP7 protein of an unknown rotavirus was detected in 5 of the 6 sows of the “rota groups” at a high level. Of these sows, each of the 3 piglets also had high levels of virus presence indicative for viremia. In the piglets of the 6^{th} sow, only low levels of the virus could be detected, around detection level. In 2 of the “control groups” the virus could not be found. In the other control group the virus could be found at a very low level in 2 out of the 3 tested piglets, around detection level. The other animals (sow and piglet) in this group were found to be negative for the virus. All rectal swabs of all tested animals were free of any other rota virus. This means that the newly found rota virus must have been responsible for the induction of rota induced disease. Phylogenetic analysis revealed that this novel virus belongs to a rotavirus B within the G12 genotype of rotavirus B (see FIG. 1; the new virus indicated as “New RotaB VP7 Spain”). Rotaviruses of this genotype have not been unambiguously found to be disease causing in the art. It is believed that the new strain is a representative of a new pathogenic sub-genus within the genus G12.

In addition, the entire coding regions of the eleven different proteins (1 protein per segment) was obtained by Illumina sequencing, revealing the sequences of SEQ ID NO. 1-22 of the new rotavirus. For the proteins of segments 2-11, sequences of the full-length coding regions, i.e. from start to stop, were obtained. For the protein of segment 1, the sequence of the partial coding region was obtained.

Genome assembly was from sequencing information obtained from serum samples. Illumina sequencing was performed as follows:

Sample “118-45 Scrum-nr-49 Sow D3004 Spain” was used for library preparation and sequencing of the segments. One hundred and ten μl of material was spun down for 10 minutes at 10,000×g and treated with TurboDNase (Thermofisher) as described (de Vries M, et al. PLOS One. 2011; 6 (1): c16118. doi: 10.1371/journal.pone.0016118), after which nucleic acids were extracted by Boom extraction method (Boom R, et al. J Clin Microbiol. 1990;28 (3): 495-503). The samples were sheared using dsDNA Fragmentase (New England Biolabs). The sheared samples were purified with

AMPure XP beads (agencourt AMPure XP PCR, Beckman Coulter) in a ratio 1:1.8 (sample: beads) to remove the enzymes. After purification the samples were end repaired with DNA polymerase I, Large (Klenow) Fragment (New England Biolabs). The end repaired samples were purified with AMPure XP beads in a ratio 1:1.8 (sample: beads) to remove the enzymes, after which the samples were A-tailed by using Klenow Fragment (3′-5′ Exo-) (New England Biolabs). The samples were purified with AMPure XP beads in a ratio 1:1.8 (sample: beads) to remove the polymerases. Bubble adaptors from the NEBNext Multiplex Oligos for Illumina (New England Biolabs) were ligated to the A-tailed samples, by use of T4 ligase. A size selection was performed by use of AMPure XP beads first in a ratio 1:0.5 (sample: beads) to ensure that most fragments with a size bigger than 400 bp were removed, followed by adding additional AMPure XP beads to the supernatant to get to a final ratio of 1:0.85 (sample: beads) to bind DNA fragments between 200-400 bp and to remove fragments smaller than 200 bp. After the size selection the Bubble adaptors were opened by using USER enzyme from the NEBNext Multiplex Oligos for Illumina (New England Biolabs). Next, a 28 cycle PCR was performed with adaptor specific primers from the NEBNext Multiplex Oligos for Illumina (New England Biolabs) and Q5 hotstart mastermix (New England Biolabs); 30 sec 98° C., and cycles of 10 sec 98°° C. and 75 sec 65° C., followed by 5 min 65° C. After PCR the samples underwent size selections by use of AMPure XP beads in a ratio 1:0.5 (sample: beads) to remove fragments with a size bigger than 400 bp, and to the supernatant additional AMPure XP beads were added to get to a final ratio of 1:0.85 (sample:beads) to bind DNA fragments between 200-400 bp and to remove fragments smaller than 200 bp. Next the concentration of the DNA was measured via Qubit dsDNA HS Assay Kit (Thermofisher), the size was checked on the bioanalyzer with a High Sensitivity DNA Analysis Kit. The library was sequenced by use of the MiSeq (Illumina) using paired end sequencing and the v2 kit (Illumina). Quality control and trimming is done with Trimmomatic 0.35 with the following settings (phred 33, LEADING: 3 TRAILING: 3 SLIDINGWINDOW: 4:15 MINLEN: 36) and assembly is done with SPAdes version 3.5.0-Darwin, with the following settings (-“careful”-“only-assembler”-“paired reads”). To have the terminal sequences of the segments (5′ and 3′) the de novo assembled contigs were aligned to segments 1 to 11 of KR052709.1, KR052710.1, KR052711.1, KR052712.1, KR052713.1, KR052714.1, KR052715.1, KR052716.1, KR052717.1, KR052718.1, KR052719.1 respectively. All assembled sequenced were checked by eye for quality by aligning to the closest relatives of each segment: AB673232.1; KF882541.1; KR052716.1; KR052717.1; KX869737.1; KX362400.1: KX869732.1; KX869733.1: KX869735.1; MG272043.1; MG272114.1.

### Example 2: Isolation of the New Rotavirus and Reinfection of Piglets with the New Rotavirus

### INTRODUCTION

The objective of this study was to propagate the novel group B rotavirus (novel RVB) field isolate of example 1 in caesarean derived, colostrum deprived (CD/CD) piglets.

The study was performed to confirm infectivity of the newly discovered virus. An animal passage of the infectious virus should result in virus with maintained virulence and high titers in the intestinal contents. In addition to that, clinical symptoms observed at the farm from which the virus originated, diarrhea, should be reproduced.

Cesarean delivery and colostrum deprivation are needed to prevent the piglets from acquiring interfering maternally derived antibodies against rotaviruses, or to become infected with rotavirus or any other virus during farrowing. Natural or other human-aided means of delivery have higher chances of unintentional infection of the piglets, which would interfere with the study.

The piglets were infected on 3 days of age since older piglets are less susceptible to a rotavirus infection. An early infection likely results in systemic viremia and infection of the gastrointestinal tract, rather than solely an infection of the gastrointestinal tract.

### Materials and Methods

For this study 5 CD/CD piglets were used. Piglets were numbered and transported to the experimental facility. For transportation, piglets were placed in specific pathogen free transport containers, and subsequently transported in a climate controlled, animal transportation vehicle. Upon arrival at the experimental facility, piglets were housed in an isolation room.

At 3 days of age (study day 0), piglets were intragastrically infected with 5 mL inoculum per piglet of the novel RVB isolate composed of fecal-derived virus obtained in Example 1.

For the preparation of infectious material, fecal contents of 7 piglets (from 3 different sows) carrying the identical novel RVB were pooled (total volume 12 mL). To activate the virus, trypsin was added to a final concentration of 2 U/mL. The material was incubated for 30 mins at 37° C., after which the volume was increased to 30 mL using EMEM-medium.

The quantity of RVB in the pooled sample was 2.26*10⁷ copies/μl based on a titration series of a plasmid containing the amplicon with known concentrations. The inoculum also contained an astrovirus, for which a qPCR diagnostic assay was set up, but without a standard curve. The Ct value of the inoculum sample was 32.75

Five (5) mLs of the inoculum were administered intragastrically per piglet using a syringe and feeding tube/urethral catheter. After applying the inoculum, 2 mLs of air were gently pushed in through the gastric tube using a syringe to empty the content of the tube completely. If necessary, the mouth of the piglet was wiped off with a paper towel containing 70% ethanol after inoculation.

Starting from the day of challenge until 3 days post infection, all piglets were observed for clinical signs of diarrhea. Prior to and following infection, individual feces samples were collected from all piglets once per day directly from the rectal opening. In case that was not successful, rectal swab samples were collected. All feces samples/rectal swab were stored immediately at −70° C. At day 3 post infection (72 h), piglets were necropsied. During necropsy, contents of the intestines and scrapings of jejunum, ileum, colon and caecum tissues were collected as two pooled samples per piglet. In addition, a small tissue sample was taken from jejunum, ileum, caecum and colon for (immune)histochemistry.

The piglets were fed Swinco opticare 2100 milk until study day 0. From study day 0 until the end of the experiment (study day 3) the piglets were fed with Swinco opticare milk Silver.

### qPCR

Reverse Transcription quantitative PCR (RT-qPCR) was performed on the fecal samples. Nucleic Acids (NA) were extracted using the Magnapure methodology (Roche).

A specific RT-qPCR for the novel Rota B virus was performed on the NA extracts.

Forward primer,:
(SEQ ID NO: 23)
5′-CAGACGATCTGATAGGGATGTATTG-3′.
Reverse primer:
(SEQ ID NO: 24)
5′-ATGTCCGTGACGTAGTATCTTC-3′.

qPCR protocol: 5 min 55° C., 5 min 95° C., [10 sec 94° C., 25 sec 58° C.]×39

The kit used for performing the qPCR was the Invitrogen SuperScript™ III Platinum™ One-Step qRT-PCR Kit. A quantitation was performed based on a titration series of a plasmid containing the amplicon with known concentrations.

An astrovirus specific RT-qPCR was performed on the NA extracts.

Forward primer:
(SEQ ID NO: 25)
5′-GTGCAGATGTGTTGGCGTATAAG-3′
Reverse primer:
(SEQ ID NO: 26)
5′-TGAAGCGTACAAACCAGGATGAG-3′

qPCR protocol: 3 min 55° C., 5 min 95° C., [15 sec 95° C., 30 sec 60° C.] x39

The kit used for performing the qPCR was the Invitrogen SuperScript™ III Platinum™ One-Step qRT-PCR Kit, catalog nr. 11732020. No quantitation based on a titration series was performed.

### RESULTS

The health status of all animals was monitored throughout the animal study. At 21 h post infection, piglets were first diagnosed with symptoms of diarrhea and decreased appetite. All piglets developed clinical symptoms similar to those observed on the index farm.

TABLE 1
Progression of clinical symptoms at different hours post infection (pi):

| Piglet, | | 33 | | | | |
|---|---|---|---|---|---|---|
| Nr. | 21 h pi | h pi | 45 h pi | 48 h pi | 58 pi | 68 h |
| 1 | Diarrhea, | Diar- | Diarrhea, | Diarrhea, | Diarrhea, | Diarrhea, |
| | decreased | rhea | decreased | decreased | decreased | decreased |
| | appetite | | appetite | appetite, | appetite, | appetite, |
| | | | | sluggish, | sluggish, | sluggish, |
| | | | | slightly | | |
| | | | | dehydrated | | |
| 2 | Diarrhea, | Diar- | Diarrhea, | Diarrhea, | Diarrhea, | Diarrhea, |
| | decreased | rhea | decreased | decreased | decreased | decreased |
| | appetite | | appetite | appetite, | appetite, | appetite, |
| | | | | sluggish, | sluggish | sluggish |
| | | | | slightly | | |
| | | | | dehydrated | | |
| 3 | Diarrhea, | Diar- | Diarrhea, | Diarrhea, | Diarrhea, | Diarrhea, |
| | decreased | rhea | decreased | decreased | decreased | decreased |
| | appetite | | appetite | appetite, | appetite, | appetite, |
| | | | | sluggish, | sluggish, | sluggish |
| | | | | dehydrated | dehydrated | |
| 4 | Diarrhea, | Diar- | Diarrhea, | Diarrhea, | Diarrhea, | Diarrhea, |
| | decreased | rhea | decreased | decreased | decreased | decreased |
| | appetite | | appetite | appetite | appetite | appetite |
| 5 | Diarrhea, | Diar- | Diarrhea, | Diarrhea, | Diarrhea, | Diarrhea, |
| | decreased | rhea | decreased | decreased | decreased | decreased |
| | appetite | | appetite | appetite, | appetite, | appetite, |
| | | | | sluggish, | sluggish | sluggish |
| | | | | slightly | | |
| | | | | dehydrated | | |

### qPCR Quantification of RVB

Nucleic acids were extracted from all fecal samples collected during the study and screened for the presence of RVB with an RVB-specific qPCR. The results are presented in Table 1 below:

| Piglet # | Sample type | Fecal inoculum | 0 hpi | 24 hpi | 48 hpi | 72 hpi |
|---|---|---|---|---|---|---|
| 1 | Rectal swab | 2.26E+07 | 0.00E+00 | 1.79E+06 | 2.71E+04 | 6.43E+05 |
| 2 | Rectal swab | 2.26E+07 | 0.00E+00 | 1.71E+07 | 8.23E+05 | 9.80E+05 |
| 3 | Rectal swab | 2.26E+07 | 0.00E+00 | 6.30E+05 | 5.52E+05 | 1.09E+04 |
| 4 | Rectal swab | 2.26E+07 | 0.00E+00 | 2.95E+05 | | 8.50E+04 |
| 5 | Rectal swab | 2.26E+07 | 0.00E+00 | 1.58E+06 | 2.65E+05 | 5.05E+04 |

No RVB was present in the fecal samples of the piglets at the start of the study. At 24 h post infection, RVB could be detected in the feces of the infected piglets. The RVB remained present in the samples taken on the following days. The sample of piglet 4 taken at 48 h was not available for analysis.

The analysis of feces or fecal swabs during the study was semi-quantitative. The feces become more watery during progression of clinical symptoms. Feces were not collected in quantified amounts.

Subsequently, a qPCR quantification of astrovirus was performed on all samples to make sure this virus, which was present in the inoculum in low concentrations as diagnosed with Next Generation Sequencing and verified using qPCR, did not have an effect on the animals during the animal study.

All fecal samples collected during the study were analysed for astrovirus presence using qPCR, but none of the fecal samples were found positive for astrovirus. Thus, no replication of this virus could be shown.

In conclusion, in vivo infectivity, replication and pathogenicity of the novel Rota B virus was confirmed in this study.

Thus, the present invention relates to the following embodiments:
1. An isolated rotavirus which is a member of the sub-species of porcine group B rotaviruses genotype G12, which in its wild type form causes diarrhea in pigs, said virus being characterized in that it has a viral genome comprising an open reading frame having a nucleotide sequence corresponding to the nucleotide sequence depicted in SEQ ID NO: 1 or a nucleotide sequence having a level of identity of at least 90% therewith.
2. The isolated virus according to embodiment 1, characterized in that the nucleotide sequence having a level of identity of at least 90% to the nucleotide sequence depicted in SEQ ID NO: 1 encodes an outer viral capsid glycoprotein VP7.
3. A nucleic acid fragment comprising an open reading frame comprising at least 100 nucleotides, characterized in that said nucleic fragment has a nucleotide sequence that corresponds to a sequence having a level of identity of at least 90% to the nucleotide sequence depicted in SEQ ID NO: 1.
4. The nucleic acid fragment according to embodiment 3, characterized in that the open reading frame encodes an outer viral capsid glycoprotein VP7.
5. The nucleic acid fragment according to embodiment 3 or 4, characterized in that the open reading frame is under the control of a heterologous promoter.
6. A recombinant protein encoded by the nucleic acid fragment according to any one of embodiments 3 to 5.
7. An outer viral capsid glycoprotein VP7 or a fragment thereof, characterized that it is encoded by a nucleic acid fragment according to any one of embodiments 3 to 5.
8. An outer viral capsid glycoprotein VP7 or a fragment thereof, characterized that it is a protein according to SEQ ID NO:2, or a protein having a level of identity of at least 90% therewith.
9. A vaccine for use in protecting against an infection caused by porcine group B rotavirus, characterized in that said vaccine comprises an immunogenically effective amount of a virus according to any one of embodiments 1 and 2, or an immunologically effective amount of a recombinant protein according to embodiment 6, or an immunologically effective amount of an outer viral capsid glycoprotein VP7 according to embodiments 7 or 8, or a nucleic acid fragment according to any of the embodiments 3 to 5, and a pharmaceutically acceptable carrier.
10. The vaccine according to embodiment 9 for use in prophylactically treating an animal.
11. An antibody or antiserum reactive with a virus according to any one of embodiments 1 to 2 or with a recombinant protein according to embodiment 6 or with an outer viral capsid glycoprotein VP7 according to embodiment 7 or 8.
12. A diagnostic test kit for the detection of antibodies reactive with a virus according to any one of embodiments 1 to 2, or with antigenic material thereof, or reactive with an outer viral capsid glycoprotein VP7 according to embodiment 7, characterized in that said test kit comprises a virus according to any one of embodiments 1 to 2 or antigenic material thereof or a recombinant protein according to embodiment 6 an outer viral capsid glycoprotein VP7 according to embodiments 7 or 8.
13. A diagnostic test kit for the detection of a virus according to any one of embodiments 1 to 2, or antigenic material thereof, or an outer viral capsid glycoprotein VP7 according to embodiments 7 or 8, characterized in that said test kit comprises antibodies reactive with a virus according to any one of embodiments 1 to 2 or antigenic material thereof or reactive with a recombinant protein according to embodiments 6 or reactive with an outer viral capsid glycoprotein VP7 according to embodiment 7 or 8.
14. A method for protecting an animal against an infection caused by porcine group B rotavirus by systemically administering a vaccine according to embodiment 9 or 10 to the animal.
15. The method according to embodiment 14, characterized in that said method comprises the mixing of a virus according to any one of embodiments 1 and 2, or a recombinant protein according to embodiment 6, or an outer viral capsid glycoprotein VP7 according to embodiments 7 or 8, or a nucleic acid fragment according to any of the embodiments 3 to 5, and a pharmaceutically acceptable carrier.
16. A virus according to any one of embodiments 1 and 2, or a recombinant protein according to embodiment 6, or an outer viral capsid glycoprotein VP7 according to embodiments 7 or 8, or a nucleic acid fragment according to any of the embodiments 3 to 5, for use in the manufacture of a vaccine for protecting an animal against an infection caused by porcine group B rotavirus.

1. An isolated rotavirus which is a member of the sub-species of porcine group B rotaviruses genotype G12, which in its wild type form causes diarrhea in pigs, said virus being characterized in that it has a viral genome comprising an open reading frame having a nucleotide sequence corresponding to the nucleotide sequence depicted in SEQ ID NO:
1 or a nucleotide sequence having at least 90% sequence identity to SEQ ID NO: 1.

2. The isolated virus according to claim 1, characterized in that the nucleotide sequence having a level of identity of at least 90% to the nucleotide sequence depicted in SEQ ID NO: 1 encodes an outer viral capsid glycoprotein VP7.

3. A nucleic acid fragment comprising an open reading frame, said open reading frame comprising at least 200 nucleotides, characterized in that said nucleic acid fragment has a nucleotide sequence that corresponds to a sequence having a level of identity of at least 90% to the nucleotide sequence depicted in SEQ ID NO: 1.

4. The nucleic acid fragment according to claim 3, characterized in that the open reading frame encodes an outer viral capsid glycoprotein VP7.

5. The nucleic acid fragment according to claim 3, characterized in that the open reading frame is under the control of a heterologous promoter.

6. A recombinant protein encoded by the open reading frame of the nucleic acid fragment according to claim 3.

7. An outer viral capsid glycoprotein VP7 or a fragment thereof, characterized that it is a protein according to SEQ ID NO:2, or a protein having a level of identity of at least 93% therewith. 8 (Currently Amended) A vaccine for use in protecting against an infection caused by porcine group B rotavirus, characterized in that said vaccine comprises an immunogenically effective amount of a virus according to claim 1, and a pharmaceutically acceptable carrier.

9. The vaccine according to claim 8 for use in prophylactically treating an animal.

10. A vaccine for use in protecting against an infection caused by porcine group B rotavirus, characterized in that said vaccine comprises an immunogenically effective amount of an outer viral capsid glycoprotein VP7 according to claim 7.

11. (canceled)

12. (canceled)

13. A method for protecting an animal against an infection caused by porcine group B rotavirus by systemically administering a vaccine according to claim 8 to the animal.

14. (canceled)

15. A virus according to any one of claim 1, for use in the manufacture of a vaccine for protecting an animal against an infection caused by porcine group B rotavirus.

16. The nucleic acid fragment according to claim 4, wherein the nucleic acid fragment is present in a nucleic acid construct or an expression vector.

17. The nucleic acid fragment according to claim 16, wherein the nucleic acid construct is a alphavirus RNA replicon particle.

18. The nucleic acid fragment according to claim 17, wherein the alphavirus RNA replicon particle is a Venezuelan Equine Encephalitis (VEE) alphavirus RNA replicon particle.

19. An alphavirus RNA replicon particle comprising a nucleic acid fragment selected from:
a nucleic acid fragment that encodes an outer viral capsid glycoprotein VP7,
a nucleic acid fragment having at least 90% nucleotide sequence identity to SEQ ID NO: 1, or
a nucleic acid fragment that encodes a outer viral capsid glycoprotein VP7 having the amino acid sequence of SEQ ID NO:2, or an amino acid sequence having at least 93% sequence identity to SEQ ID NO: 2.

20. The alphavirus RNA replicon particle of claim 19, wherein the alphavirus RNA replicon particle is a Venezuelan Equine Encephalitis (VEE) alphavirus RNA replicon particle.

21. The nucleic acid fragment according to claim 16, for use in the manufacturer of a vaccine for protecting an animal against an infection caused by a porcine group B rotavirus.

22. An immunogenic composition comprising the alphavirus RNA replicon of claim 19 and a pharmaceutically acceptable carrier.

23. A vaccine to aid the prevention of disease due to a porcine group B rotavirus, comprising the nucleic acid fragment of claim 18 and a pharmaceutically acceptable carrier.
